(19) United States
(12) Patent Application Publication
(10) Pub. No.: US 2024/0252269 A1
(43) Pub. Date: Aug. 1, 2024

(54) MEDICAL MANIPULATOR SYSTEM AND ADAPTER DEVICE

(71) Applicant: SONY GROUP CORPORATION, TOKYO (JP)

(72) Inventor: KAZUHITO WAKANA, TOKYO (JP)

(21) Appl. No.: 18/006,575

(22) PCT Filed: Jun. 9, 2021
(86) PCT No.: PCT/JP2021/021918

§ 371 Date: Jan. 23, 2023

(30) Foreign Application Priority Data
Jul. 31, 2020 (JP) 2020-131253

(51) Int. Cl. A61B 34/00; A61B 46/10

(52) U.S. Cl. CPC A61B 34/70; A61B 46/10

(57) ABSTRACT

Provided is a medical manipulator system that prevents a clean region and an unclean region from being mixed between a surgical instrument subjected to sterilization processing and a drive part not subjected to the sterilization processing. The medical manipulator system includes a surgical instrument unit including a surgical instrument at a front end, a drive unit that drives the surgical instrument, and an adapter that attaches the surgical instrument unit to the drive unit. The adapter includes: a translatory transmission part that transmits a driving force generated by the drive unit to the surgical instrument unit; and a drip-proof part that separates the surgical instrument unit side and the drive unit side in the translatory transmission part. The drip-proof part includes an air chamber between the surgical instrument unit side and the drive unit side in the translatory transmission part.

### TECHNICAL FIELD

The technology (hereinafter, “the present disclosure”) disclosed in the present specification relates to a medical manipulator system including a surgical instrument and a drive part that drives the surgical instrument, and an adapter device that attaches the surgical instrument to the drive part.

### BACKGROUND ART

Recent progress in robotics technology is remarkable, and the robotics technology has widely permeated work sites in various industrial fields. For example, in the medical field, surgical robots have been widespread. A master-slave surgical robot is configured such that an operator such as a surgeon manipulates one or a plurality of surgical instruments provided in a slave device from a master side. The slave device includes an articulated arm having a multi-link structure and a surgical instrument and an observation device attached to a front end of the arm, and is configured to be manipulated by the operator such as the surgeon from the master side. Examples of the surgical instrument include forceps, a pneumoperitoneum tube, an energy treatment instrument, tweezers, a retractor, and the like. Examples of the observation device include an endoscope, a microscope, and the like.

Since the surgical instrument is used in a treatment on a body cavity, a body surface, or the like, it is strongly desired for a front end of the surgical instrument to have multiple degrees of freedom, a small diameter, a small size, and light weight. Specifically, it is desirable for the front end of the surgical instrument to have three or more degrees of freedom in total, that is, two degrees of freedom for rotation and a degree of freedom for opening and closing. Furthermore, a driving method using a wire is often applied to the manipulation of the surgical instrument front end in order to for size reduction.

Furthermore, the surgical instrument is brought into direct contact with a body cavity, and thus, needs to be subjected to sterilization processing prior to use. As a method for the sterilization processing, there are various methods such as high-pressure steam sterilization (autoclave sterilization) in which microorganisms are sterilized by high-temperature and high-pressure saturated steam, and EOG gas sterilization in which microorganisms are sterilized by alkylation using ethylene oxide gas (EOG). In general, an arm and a drive part that drives a front end of the arm do not often have structures that can withstand sterilization processing. Thus, the sterilization processing is performed separately from a portion that requires the sterilization processing such as the surgical instrument and a portion that does not has a structure capable of withstanding the sterilization processing such as the drive part. However, in a case where the surgical instrument subjected to the sterilization processing is attached to the front end of the arm, a clean region where the sterilization processing has been performed and an unclean region where the sterilization processing has not been performed are likely to be mixed so that the clean region is contaminated during the attachment, or at the time of driving the surgical instrument during surgery, for example.

For example, a sterile surgical adapter is proposed (see Patent Document 1). The sterile surgical adapter has a structure in which a surgical instrument with four degrees of freedom is decoupled from the arm, and is configured to connect a capstan on the surgical instrument side and a capstan on the arm side with a drip-proof bearing so as to separate a clean region and an unclean region. However, the surgical instrument connected by the sterile surgical adapter includes a rotation mechanism, and thus, a proximal end on the surgical instrument side becomes large.

Furthermore, there is proposed a power transmission adapter that makes connection by a bellows-shaped connection part with a clean region and an unclean region being separated (see Patent Document 2). In a case where a surgical instrument and a drive part are connected using the power transmission adapter, there is a concern that a bellows of the connection part hinders translatory movement of the surgical instrument or a wire is loosened when the surgical instrument is detached.

Furthermore, there is proposed a surgical power transmission adapter that includes rods 222a and 222b having a first region in contact with a clean region that is subjected to sterilization processing and a second region in contact with an unclean region that is not subjected to the sterilization processing, and sets ranges of translatory motions of the rods 222a and 222b in such a manner that the first region is placed in the clean region and the second region is placed in the unclean region even if the rods 222a and 222b make the translatory motions, thereby preventing the clean region and the unclean region from being mixed (see Patent Document 3). Since this surgical power transmission adapter has a structure for performing drip-proofing only with a fit tolerance, it is not easy to manufacture and assemble components, and there is a concern that it is difficult to completely perform the drip-proofing.

### CITATION LIST

### Patent Document

Patent Document 1: Japanese Patent Application Laid-Open No. 2015-91331
Patent Document 2: Japanese Patent Application Laid-Open No. 2018-143426
Patent Document 3: Japanese Patent Application Laid-Open No. 2012-55377

### SUMMARY OF THE INVENTION

### Problems to be Solved by the Invention

An object of the present disclosure is to provide a medical manipulator system that includes a surgical instrument and a drive part driving the surgical instrument and prevents a clean region and an unclean region from being mixed between the surgical instrument subjected to sterilization processing and the drive part not subjected to the sterilization processing, and an adapter device that prevents the clean region and the unclean region from being mixed and attaches the surgical instrument to the drive part.

### Solutions to Problems

The present disclosure has been made in view of the problems described above, and a first aspect thereof is a medical manipulator system including:
a surgical instrument unit that includes a surgical instrument at a front end;
a drive unit that drives the surgical instrument; and
an adapter that attaches the surgical instrument unit to the drive unit,
the adapter including: a translatory transmission part that transmits a driving force generated by the drive unit to the surgical instrument unit; and a drip-proof part that separates a side of the surgical instrument unit and a side of the drive unit in the translatory transmission part.

The drip-proof part has a structure in which two elastic bodies each having a two-fold structure are arranged to face each other. An air chamber is provided between the surgical instrument unit side and the drive unit side in the translatory transmission part by connecting both ends with the translatory transmission part to separate the surgical instrument unit side and the drive unit side.

Furthermore, a second aspect of the present disclosure is an adapter device having one end to which a drive unit is attached and another end to which a surgical instrument unit is attached, the adapter device including:
a translatory transmission part that transmits a driving force generated by the drive unit to the surgical instrument unit; and
a drip-proof part that separates a side of the surgical instrument unit and a side of the drive unit in the translatory transmission part.

### Effects of the Invention

According to the present disclosure, it is possible to provide the medical manipulator system that separates the clean region and the unclean region by the adapter connecting the surgical instrument and the drive part, and the adapter device that connects the surgical instrument and the drive part in a state in which the clean region and the unclean region are separated.

Note that the effects described in the present specification are merely examples, and effects brought by the present disclosure are not limited thereto. Furthermore, there is also a case where the present disclosure further provides additional effects in addition to the effects described above.

Still other objects, characteristics and advantages of the present disclosure will become apparent from a detailed description based on embodiments as described later and accompanying drawings.

### BRIEF DESCRIPTION OF DRAWINGS

FIG. 1 is a diagram illustrating an example of a functional configuration of a surgical support system 100.

FIG. 2 is a diagram illustrating an example of an external configuration of a medical arm device 110.

FIG. 3 is a diagram illustrating an example of an external configuration of a surgical manipulation device 300.

FIG. 4 is a diagram illustrating a configuration example of a surgical instrument unit 400.

FIG. 5 is a diagram illustrating an enlarged front end part 401 of the surgical instrument unit 400.

FIG. 6 is a diagram illustrating an example of a freedom degree configuration of the surgical instrument unit 400.

FIG. 7 is a diagram illustrating an example of an operation of a wrist element WE about a first axis.

FIG. 8 is a diagram illustrating an example of an operation of an end effector about a second axis.

FIG. 9 is a diagram illustrating an example of an operation of the end effector about the second axis.

FIG. 10 is a diagram illustrating a configuration example of a surgical instrument device 1000.

FIG. 11 is a diagram illustrating a configuration example of the surgical instrument device 1000.

FIG. 12 is a diagram illustrating a cross section of the surgical instrument device 1000.

FIG. 13 is a diagram illustrating a cross section of the surgical instrument device 1000.

FIG. 14 is a diagram illustrating an enlarged cross section of a vicinity of an adapter 1002 immediately after attachment.

FIG. 15 is a diagram illustrating an enlarged cross section of the vicinity of the adapter 1002 during operation.

FIG. 16 is a diagram illustrating a cross section (immediately after attachment) of the adapter 1002.

FIG. 17 is a diagram illustrating a cross section (operating state) of the adapter 1002.

FIG. 18 is a diagram illustrating a cross section (immediately after attachment) of the adapter 1002.

FIG. 19 is a diagram illustrating a cross section (operating state) of the adapter 1002.

FIG. 20 is a diagram illustrating a cross section (operating state) of the adapter 1002.

FIG. 21 is a diagram illustrating a change of an air chamber accompanying movement of a linear shaft B in a longitudinal-axis direction.

FIG. 22 is a diagram illustrating a change of the air chamber accompanying the movement of the linear shaft B in the longitudinal-axis direction.

FIG. 23 is a diagram illustrating a change of the air chamber accompanying the movement of the linear shaft B in the longitudinal-axis direction.

FIG. 24 is a diagram illustrating an example of an external configuration of the adapter 1002.

FIG. 25 is a diagram illustrating a shape of a rear end portion of a linear shaft C and a shape of a front end portion of the linear shaft B.

FIG. 26 is a diagram illustrating an appearance (a front surface, a side surface, a rear surface, and a three-dimensional view) of a locking device 2600.

FIG. 27 is a diagram illustrating an external configuration of the locking device 2600 in a lock-off state.

FIG. 28 is a diagram illustrating an external configuration of the locking device 2600 in the lock-off state.

FIG. 29 is a diagram illustrating an external configuration of the locking device 2600 in a lock-on state.

FIG. 30 is a diagram illustrating an external configuration of the locking device 2600 in the lock-on state.

FIG. 31 is a diagram illustrating a cross section of the locking device 2600 in the lock-on state.

FIG. 32 is a diagram illustrating a procedure for attaching the adapter 1002 to a drive unit 1003.

FIG. 33 is a diagram illustrating a procedure for attaching the adapter 1002 to the drive unit 1003.

FIG. 34 is a diagram illustrating a procedure for attaching the adapter 1002 to the drive unit 1003.

FIG. 35 is a diagram illustrating a procedure for attaching the adapter 1002 to the drive unit 1003.

FIG. 36 is a diagram illustrating a procedure for attaching a surgical instrument unit 1001 to the adapter 1002.

FIG. 37 is a diagram illustrating a procedure for attaching the surgical instrument unit 1001 to the adapter 1002.

FIG. 38 is a diagram illustrating a procedure for attaching the surgical instrument unit 1001 to the adapter 1002.

FIG. 39 is a diagram illustrating a procedure for attaching the surgical instrument unit 1001 to the adapter 1002.

FIG. 40 is a diagram illustrating a procedure for detaching the surgical instrument unit 1001 from the adapter 1002.

FIG. 41 is a diagram illustrating a procedure for detaching the surgical instrument unit 1001 from the adapter 1002.

FIG. 42 is a diagram illustrating a procedure for detaching the surgical instrument unit 1001 from the adapter 1002.

FIG. 43 is a diagram illustrating a procedure for detaching the surgical instrument unit 1001 from the adapter 1002.

FIG. 44 is a diagram illustrating a procedure for detaching the adapter 1002 from the drive unit 1003.

FIG. 45 is a diagram illustrating a procedure for detaching the adapter 1002 from the drive unit 1003.

FIG. 46 is a diagram illustrating a procedure for detaching the adapter 1002 from the drive unit 1003.

FIG. 47 is a diagram illustrating a procedure for detaching the adapter 1002 from the drive unit 1003.

### MODE FOR CARRYING OUT THE INVENTION

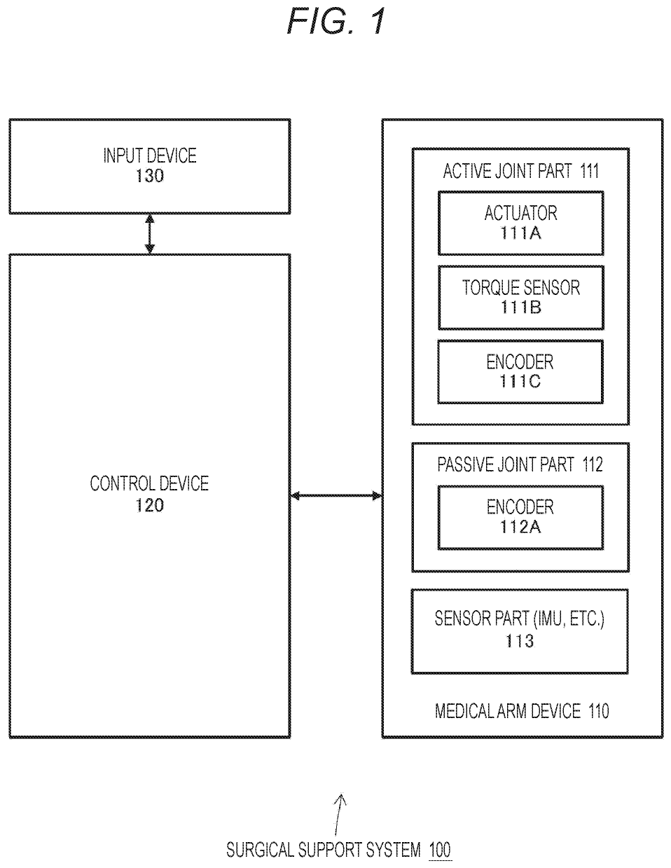

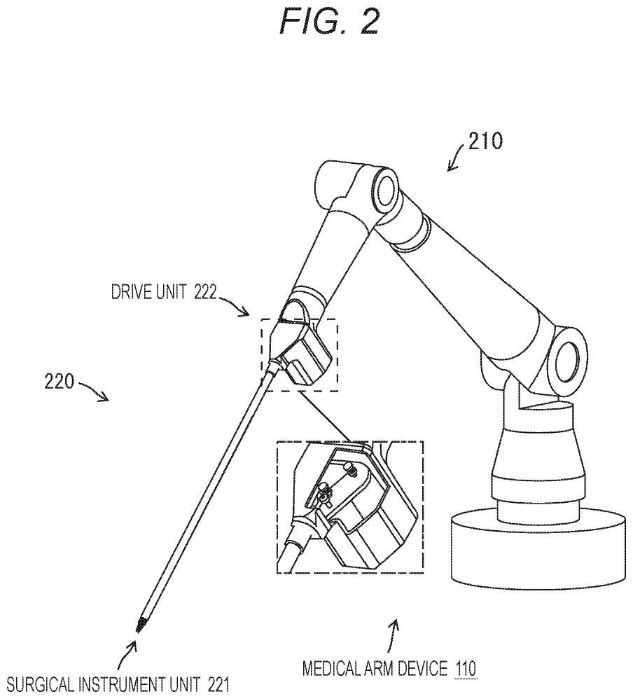

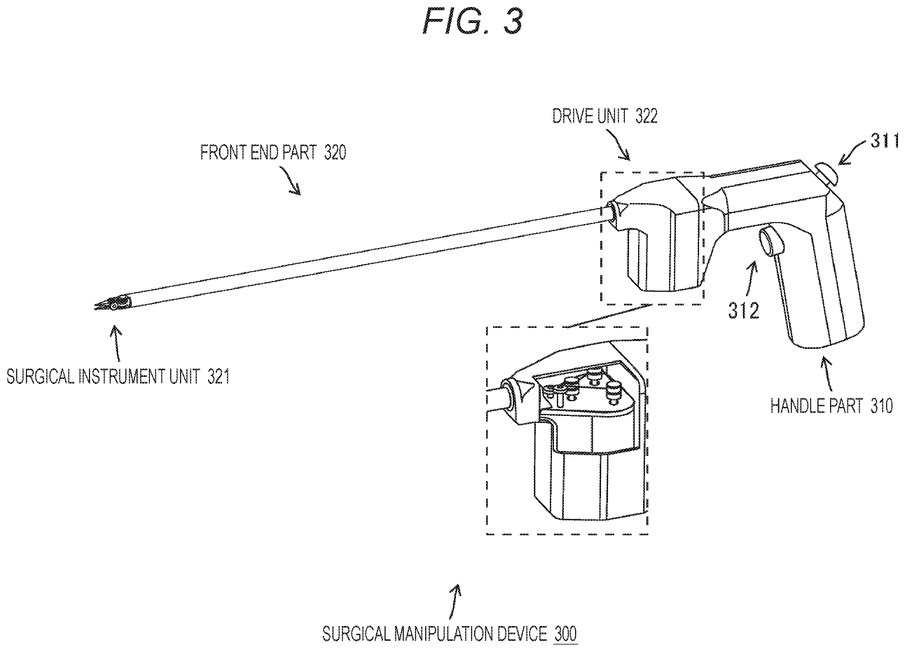

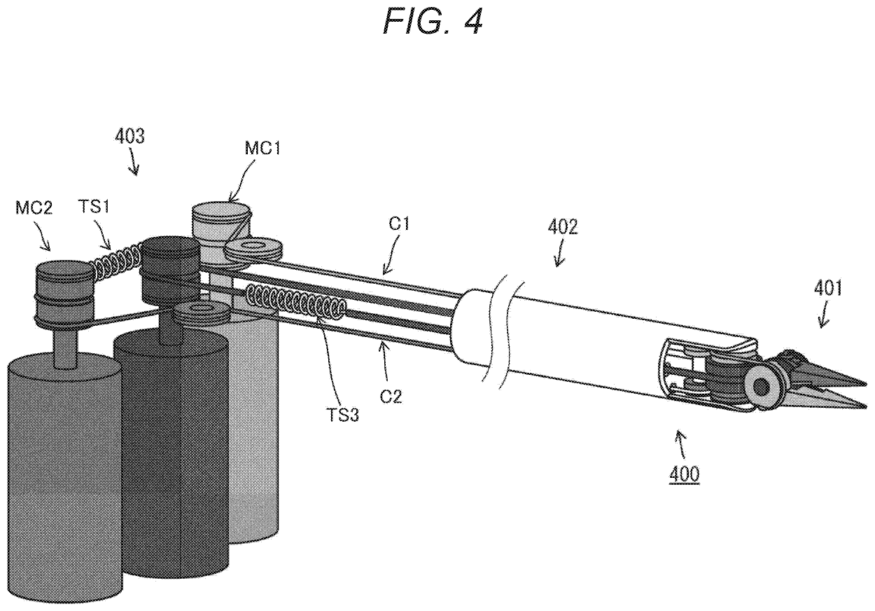

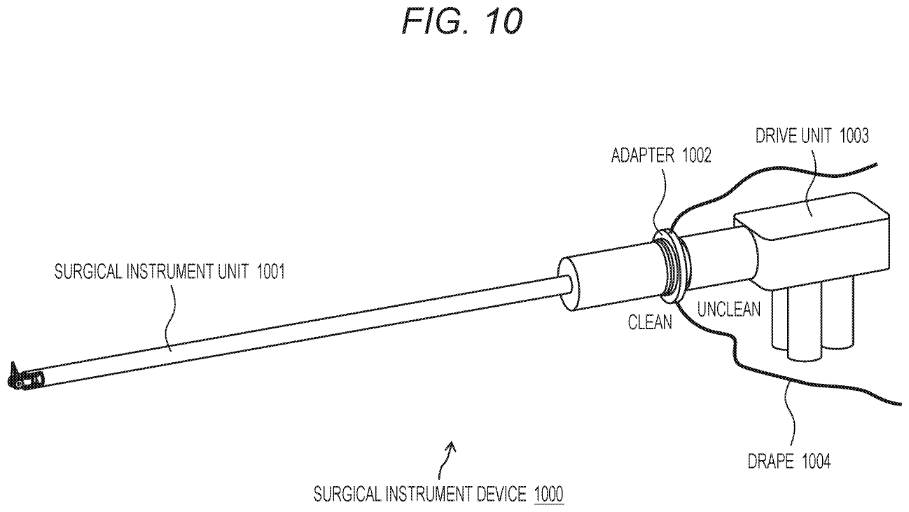

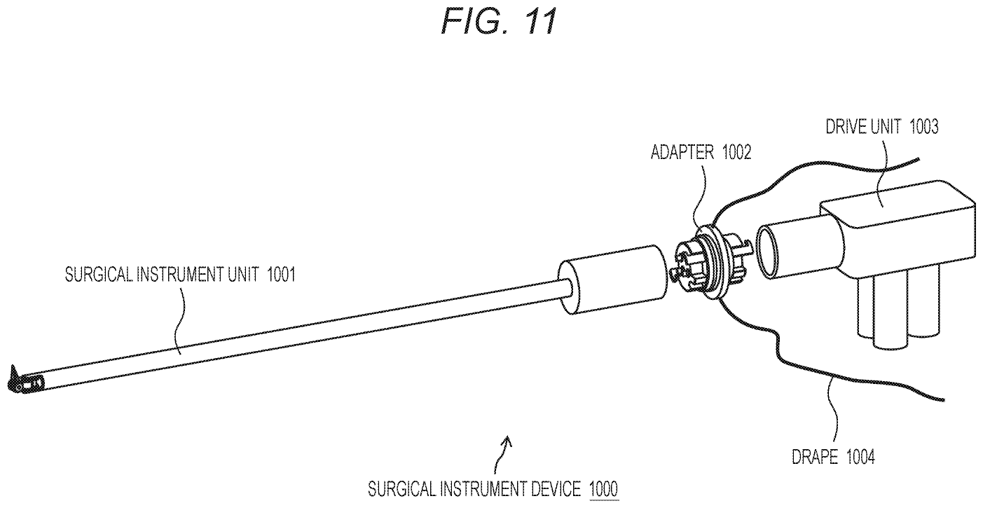

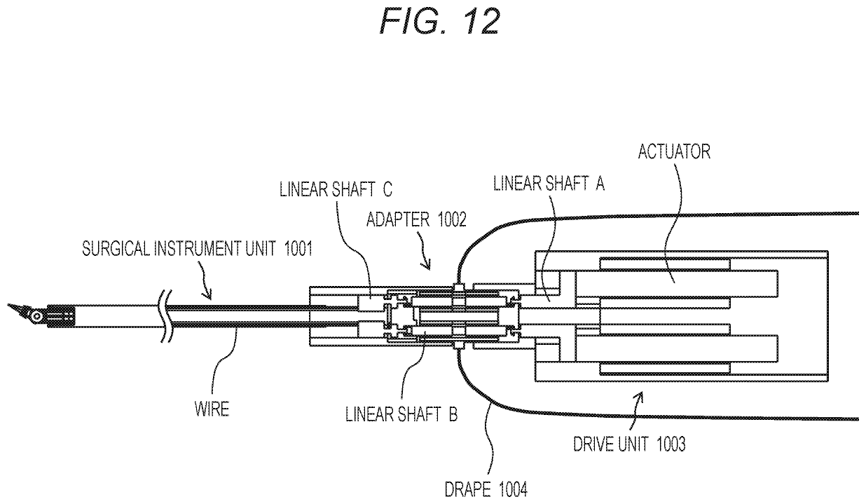

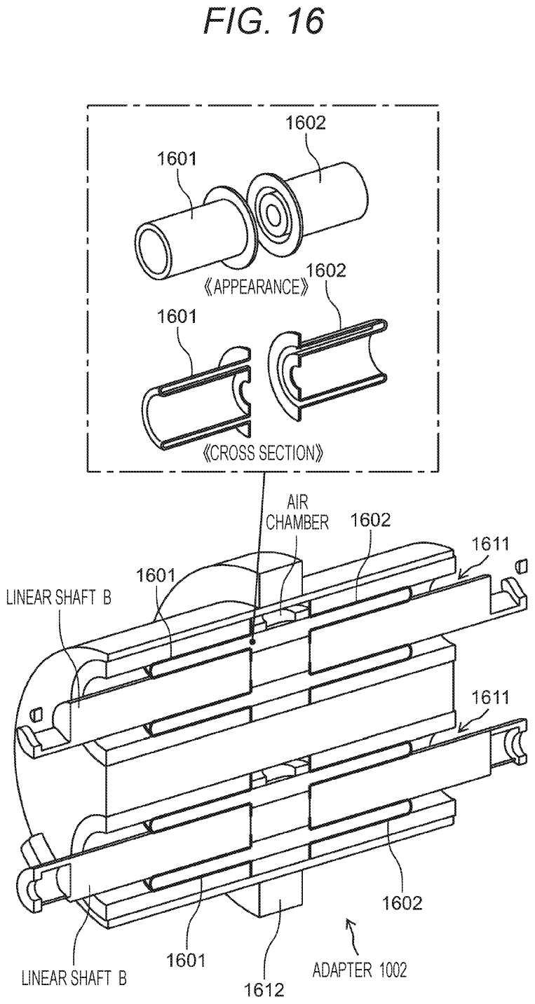

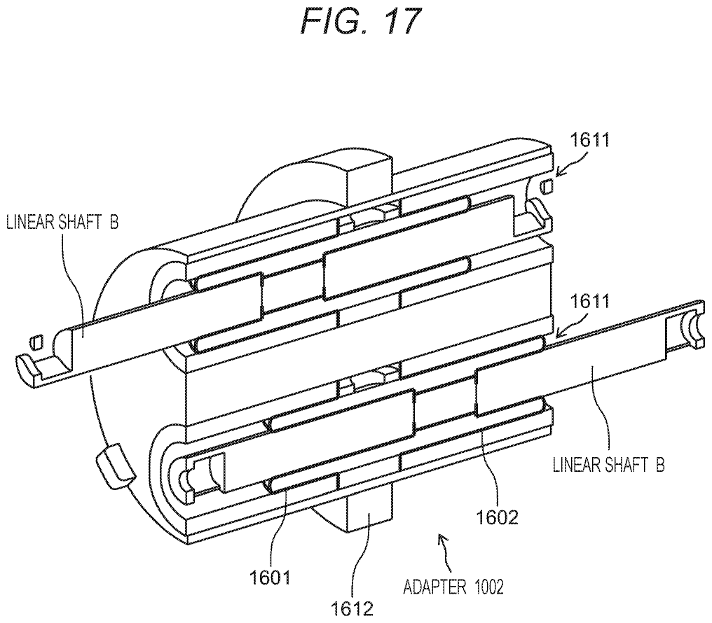

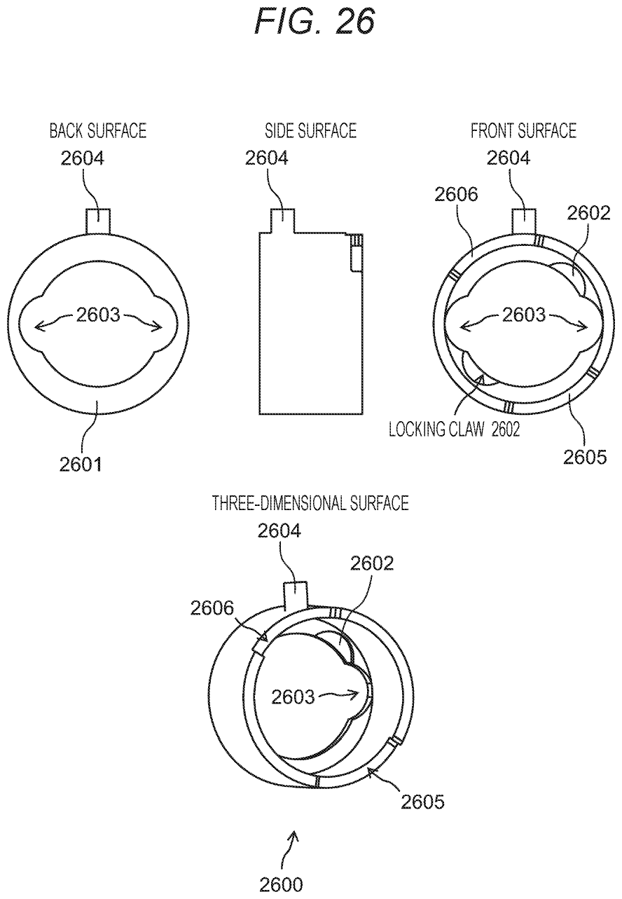

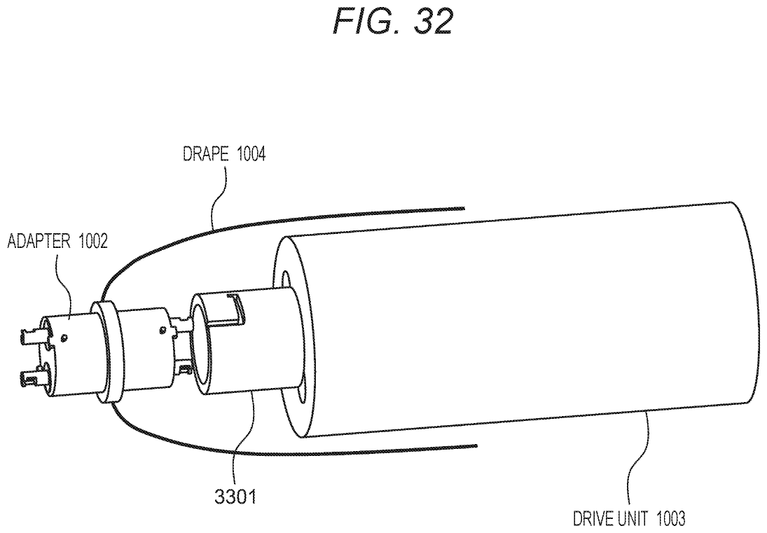

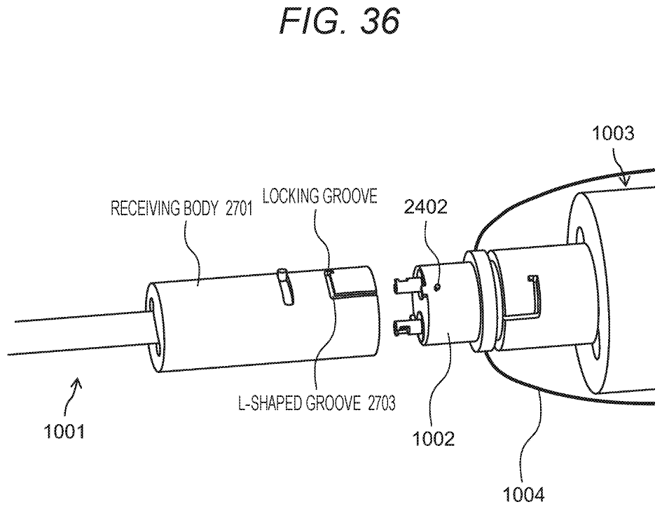

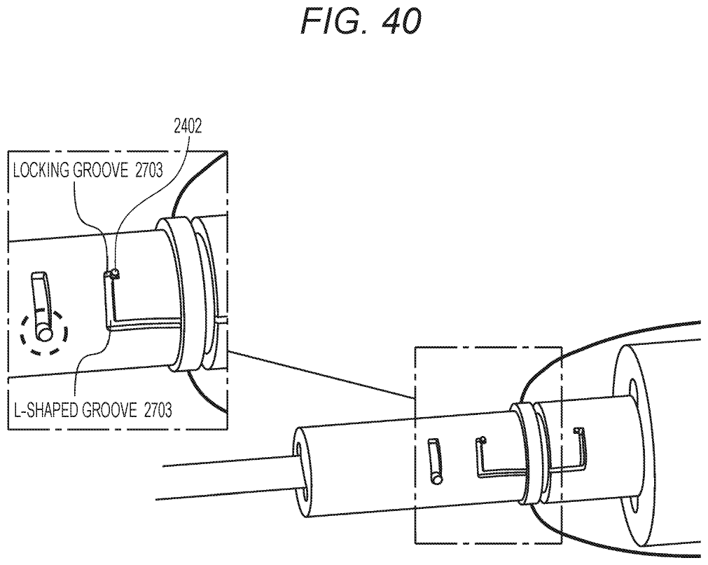

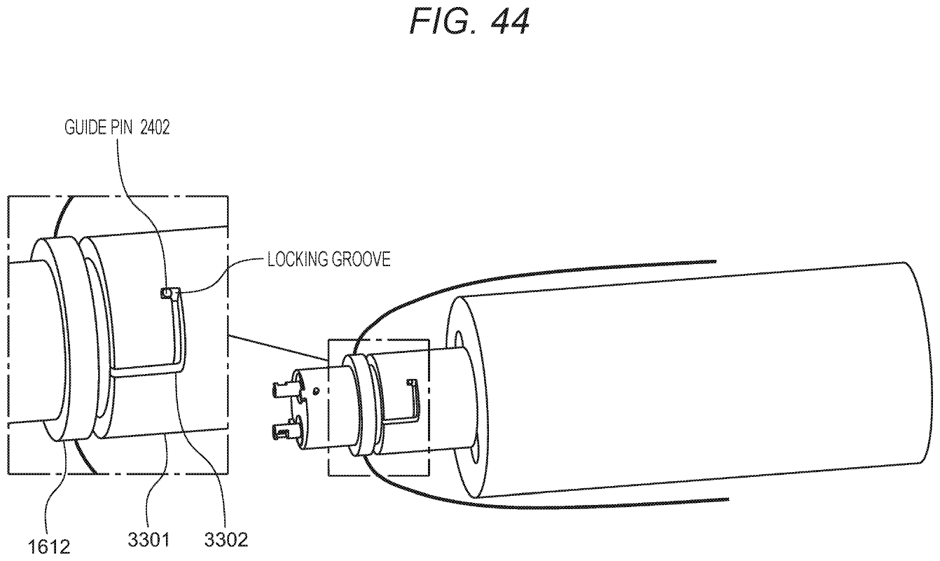

Hereinafter, the present disclosure will be described in the following order with reference to the drawings.
A. Surgical Support System (FIG. 1)
B. External Configuration of Medical Arm Device (FIG. 2)
C. External Configuration of Surgical Manipulation Unit (FIG. 3)
D. Drive Mechanism of Surgical Instrument Unit (FIGS. 4 to 9)
E. Surgical Instrument Device Using Drip-Proof Adapter
   E-1. Overall Configuration of Surgical Instrument Device (FIGS. 10 to 11)
   E-2. Translatory Transmission Structure of Adapter (FIGS. 12 to 15)
   E-3. Drip-Proof Structure of Adapter (FIGS. 16 to 25)
   E-4. Locking Device of Surgical Instrument Unit (FIGS. 26 to 31)
F. Attachment and Detachment Procedure
   F-1. Attachment of Adapter to Drive Unit (FIGS. 32 to 35)
   F-2. Attachment of Surgical Instrument Unit to Adapter (FIGS. 36 to 39)
   F-3. Detachment of Surgical Instrument Unit from Adapter (FIGS. 40 to 43)
   F-4. Detachment of Adapter from Drive Unit (FIGS. 44 to 47)
G. Modified Examples
   G-1. Regarding Number of Translatory Transmission Mechanisms
   G-2. Regarding Locking Device
   G-3. Regarding adapter
   G-4. Regarding Actuator
   G-5. Identification of Surgical Instrument Unit
   G-6. Regarding Operation
H. Effects

### A. Surgical Support System

FIG. 1 schematically illustrates an example of a functional configuration of a surgical support system 100 to which the present disclosure can be applied. The illustrated surgical support system 100 includes a medical arm device 110, a control device 120, and an input device 130. In a case where the surgical support system 100 is a master-slave surgical robot, the input device 130 corresponds to a controller manipulated by an operator such as a surgeon, the control device 120 corresponds to a master device that outputs a command to a slave device on the basis of a manipulation of the operator, and the medical arm 110 corresponds to the slave device that operates according to the command output from the master device.

The medical arm device 110 is, for example, a “medical manipulator system” including an articulated arm having a multi-link structure and a surgical instrument attached to a front end of the arm. The arm has three or more degrees of freedom for determining positions and postures of a front end part. Furthermore, the surgical instrument at the front end is, for example, forceps, and has three or more degrees of freedom in total including two degrees of freedom for rotation and a degree of freedom for opening and closing. Since the surgical instrument is used in a treatment on a body cavity, a body surface, or the like, a driving method using a wire is applied to the manipulation of a front end of the surgical instrument in order for size reduction. Meanwhile, a detailed structure of the surgical instrument will be described later. In FIG. 1, functions of the medical arm device 110 are abstracted such that joints connecting links and joints of the surgical instrument are classified into two types of an active joint part 111 and a passive joint part 112, and include a sensor part 113.

The active joint part 111 includes an actuator 111A such as a rotary motor that drives a joint, a torque sensor 111B that detects a torque acting on a joint, and an encoder 111C that measures a rotating angle of a joint. Furthermore, the passive joint part 112 includes an encoder 112A that measures a joint angle. The sensor part 113 includes various sensors arranged outside the joint parts such as an inertial measurement unit (IMU) and a contact sensor that detects a contact force acting on a medical instrument attached to the front end of the robot arm.

The control device 120 generates a target o of the medical arm device 110 on the basis of an instruction input by an operator such as a surgeon, via the input device 130, and controls driving of the medical arm device 110 according to a predetermined control method such as position control or force control. Specifically, the control device 120 calculates the amount of control of the actuator 111A of the active joint part 111 according to a predetermined control method and supplies a drive signal, and performs feedback control of the actuator 111A on the basis of sensor signals from the torque sensor 111B, the encoder 111C, and the sensor part 113. The control device 120 includes, for example, a processor such as a central processing unit (CPU), a local memory thereof, and the like and executes a predetermined program loaded on the local memory by the processor.

The control device 120 and the medical arm device 110 may be connected to each other wirelessly or by an electrical signal wire corresponding to communication of an electrical signal, an optical fiber corresponding to optical communication, or a composite wire thereof.

### B. External Configuration of Medical Arm Device

FIG. 2 illustrates an example of an external configuration of the medical arm device 110 to which the present disclosure is applied. The illustrated medical arm device 110 includes an arm 210 having a multi-link structure and a front end part 220 supported by a distal end of the arm 210.

The front end part 220 includes a surgical instrument unit 221 and a drive unit 222 that drives the surgical instrument unit 221. A driving method using a wire is applied to the manipulation of the surgical instrument unit 221 in order for size reduction. Therefore, a driving force generated by an actuator in the drive unit 222 is transmitted to the surgical instrument unit 221 using the wire (not illustrated). In the case of forceps, for example, the surgical instrument unit 221 has three degrees of freedom in total including two degrees of freedom for rotation and a degree of freedom for opening and closing. Furthermore, the drive unit 222 is provided with three actuators respectively corresponding to the degrees of freedom. Details of a drive mechanism of the surgical instrument unit 221 and the drive unit 222 will be described later.

In the present disclosure, it is assumed that the surgical instrument unit 221 is replaceable. The surgical instrument unit 221 is brought into direct contact with a body cavity, and thus, needs to be subjected to sterilization processing prior to use. On the other hand, the drive unit 222 does not have a structure capable of withstanding the sterilization processing. Thus, the surgical instrument unit 221 is separated from the drive unit 222 and subjected to the sterilization processing. Furthermore, in the present disclosure, the surgical instrument unit 221 is attached to the drive unit 222 via a drip-proof adapter including a translatory transmission mechanism in order to maintain a state in which a clean region and an unclean region are separated when the surgical instrument unit 221 is attached to the drive unit 222 and driven or the like. In FIG. 2, illustration of the drip-proof adapter is omitted for simplification of the drawing. Details of the drip-proof adapter will be described later.

Note that the arm 210 may be any mechanism robot among a polar coordinate robot, a cylindrical coordinate robot, a rectangular coordinate robot, a vertical articulated robot, a horizontal articulated robot, a parallel link robot, a remote center of motion (RCM) robot, and the like. Furthermore, from the viewpoint of compactness of a mechanism, ease of generating a pivot motion at a trocar site, and the like, the vertical articulated arm or the remote center of motion (RCM) arm that implements pivot (fixed point) motion by arranging a remote rotation center at a position away from a drive rotation center may be used as the arm 210.

### C. External Configuration of Surgical Manipulation Device

An adapter, which connects a surgical instrument and a drive part with a clean region and an unclean region being separated, according to the present disclosure can be applied not only to the medical arm device (see FIG. 2) but also to a surgical manipulation device manipulated by a surgeon holding the adapter in a hand. FIG. 3 illustrates an example of an external configuration of a surgical manipulation device 300 to which the present disclosure is applied. The illustrated surgical manipulation device 300 includes a handle part 310 that is directly held in a hand and manipulated by a user (surgeon) and a front end part 320 supported by a distal end of the handle part 310.

The front end part 320 includes a surgical instrument unit 321 and a drive unit 322 that drives the surgical instrument unit 321. A driving method using a wire is applied to the manipulation of the surgical instrument unit 321 in order for size reduction. Therefore, a driving force generated by an actuator in the drive unit 322 is transmitted to the surgical instrument unit 321 using the wire (not illustrated). In the case of forceps, for example, the surgical instrument unit 321 has three degrees of freedom in total including two degrees of freedom for rotation and a degree of freedom for opening and closing. Furthermore, the drive unit 322 is provided with three actuators respectively corresponding to the degrees of freedom. Details of a drive mechanism of the surgical instrument unit 321 and the drive unit 322 will be described later.

The handle part 310 may include, for example, a joystick 311 that is configured to instruct a posture of the surgical instrument unit 321 in any direction and can be manipulated with a thumb. Furthermore, the handle part 310 may include a button 312 that is configured to instruct opening and closing manipulations of the surgical instrument unit 321 and can be manipulated with an index finger. A controller (not illustrated) is mounted inside the handle part 310. The controller calculates a rotation angle or an opening/closing angle of the surgical instrument unit 321 for each degree of freedom according to the amount of manipulation of the joystick 311 or the button 312, converts the calculated angle into the amount of rotation of each motor, and outputs a control signal to the surgical instrument unit drive part 322.

In the present disclosure, it is assumed that the surgical instrument unit 321 is replaceable. The surgical instrument unit 321 is brought into direct contact with a body cavity, and thus, needs to be subjected to sterilization processing prior to use. On the other hand, the drive unit 322 does not have a structure capable of withstanding the sterilization processing. Thus, the surgical instrument unit 321 is separated from the drive unit 322 and subjected to the sterilization processing. Furthermore, in the present disclosure, the surgical instrument unit 321 is attached to the drive unit 322 via a drip-proof adapter including a translatory transmission mechanism in order to maintain a state in which a clean region and an unclean region are separated when the surgical instrument unit 321 is attached to the drive unit 322 and driven or the like. In FIG. 3, illustration of the drip-proof adapter is omitted for simplification of the drawing. Details of the drip-proof adapter will be described later.

### D. Drive Mechanism of Surgical Instrument Unit

A surgical instrument unit to which the present disclosure is applied is, for example, forceps, has three or more degrees of freedom in total including two degrees of freedom for rotation and a degree of freedom for opening and closing, and is driven using a driving force generated by a drive unit. Furthermore, a driving method using a wire is applied to the manipulation of the surgical instrument unit in order for size reduction, and the drive unit pulls the surgical instrument unit via the wire. In this section, a drive mechanism of the surgical instrument unit using the wire driving method will be described.

FIG. 4 illustrates a configuration example of a surgical instrument unit 400 to which the present disclosure is applied. The surgical instrument unit 400 includes an opening and closing mechanism 401 including a pair of opposing jaw members at the frontmost end part. The surgical instrument unit 400 is coupled with a drive unit 403 including three actuators via a hollow shaft 402 having a longitudinal axis. Note that the surgical instrument unit 400 is attachable to and detachable from the drive unit 403 in a state in which a clean region and an unclean region are separated via a drip-proof adapter (not illustrated) provided near the middle of the shaft 402. Meanwhile, a structure of the drip-proof adapter is not described in this section.

The surgical instrument unit 400 includes a wrist element WE capable of turning about a first axis parallel to a yaw axis with respect to the shaft 402, and an end effector that is opened and closed with a second axis, parallel to a pitch axis, as an opening and closing axis at a front end of the wrist element WE as described later. Meanwhile, the second axis is arranged at a position offset from the first axis. The end effector includes the pair of opposing jaw members turning about the second axis to be opened and closed. Furthermore, the drive unit 403 includes one actuator that drives the wrist in the surgical instrument unit 400 and two actuators that drive the jaw members, respectively. These actuators are attached to a vicinity of a rear end (proximal end) of the shaft 402 by a base member (not illustrated).

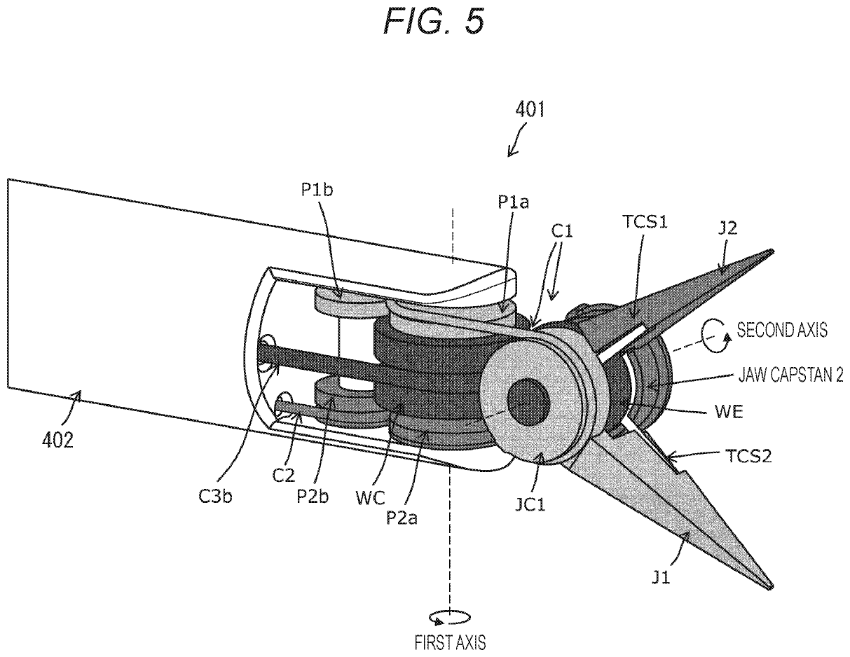

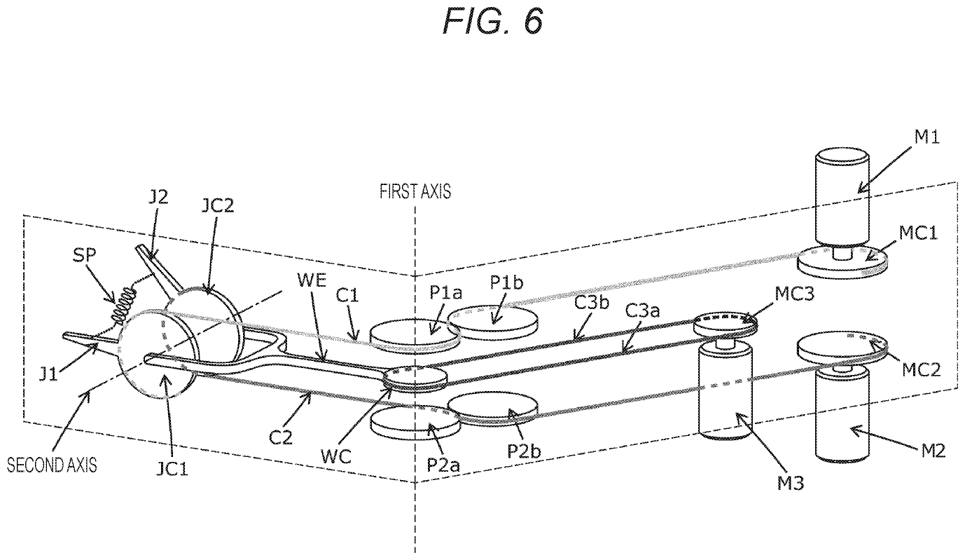

FIG. 5 illustrates an enlarged front end part 401 of the surgical instrument unit 400. Furthermore, FIG. 6 illustrates an example of a freedom degree configuration of the surgical instrument unit 400.

The front end part 401 of the surgical instrument unit 400 includes the wrist element WE and an openable and closable end effector, and the end effector includes the pair of opposing jaw members, that is, a first jaw member J1 and a second jaw member J2. The wrist element WE is supported near the base so as to be turnable about the first axis parallel to the yaw axis at a front end (distal end) of the shaft 402. Furthermore, the first jaw member J1 and the second jaw member J2 are supported so as to be turnable about the second axis parallel to the pitch axis at the front end of the wrist element WE. The first jaw member J1 and the second jaw member J2 are opened and closed by changing an opening angle with the second axis as the opening and closing axis.

The drive unit 403 includes a first motor M1 used for driving the first jaw member J1, a second motor M2 used for driving the second jaw member J2, and a motor M3 used for driving the wrist element WE. These motors M1 to M3 have output shafts to which motor capstans MC1, MC2, and MC3 serving as drive capstans are attached, respectively. Then, these motors M1 to M3 are supported at an end part (proximal end) of the shaft 402 by a base member (not illustrated).

A wrist capstan WC having the first axis as the rotation axis is provided near the base of the wrist element WE. Furthermore, a third wire inserted into the shaft 402 is wound around the wrist capstan WC and the third motor capstan MC3. Then, a driving force generated by the third motor M3 is transmitted by the third wire 3 to achieve the turning operation of the wrist element WE about the first axis.

In the example illustrated in FIG. 6, the third wire includes a wire C3a for a forward path and a wire C3b for a backward path, but has a wire loop configuration that is looped between the third motor capstan MC3 on a driving side and the wrist capstan WC on an output side. When the second motor M3 is rotated, a difference in tension is generated between the wire C3a for the forward path and the wire C3b for the backward path depending on the rotation direction thereof, and thus, a rotational torque based on the difference in tension acts on the wrist capstan WC, and the wrist element WE turns about the first axis. Therefore, the wrist element WE can be turned about the first axis by antagonistic control of the wire C3a for the forward path and the wire C3b for the backward path performed by the third motor M3. As illustrated in FIG. 4, for example, a tension spring TS3 that applies pre-tension to the wire C3b side is inserted into the third wire including the wire C3a and the wire C3b in order to prevent bending. However, it may be configured to apply the pre-tension using an additional idler pulley.

The first jaw member J1 is supported by the wrist element WE near the base so as to be turnable about the second axis. Similarly, the second jaw member J2 is supported by the wrist element WE near the base so as to be turnable about the second axis. Therefore, the opening or closing operation of the end effector is implemented by turning each of the first jaw member J1 and the second jaw member J2 about the second axis such that the opening angle therebetween increases or decreases (in other words, a difference in angle about the second axis between the first jaw member J1 and the second jaw member J2 changes). Furthermore, the turning operation of the end effector including the first jaw member J1 and the second jaw member J2 about the second axis is implemented by simultaneously turning the first jaw member J1 and the second jaw member J2 about the second axis while the opening angle therebetween is maintained constant (in other words, such that the sum of angles about the second axis of the first jaw member J1 and the second jaw member J2 changes).

A first jaw capstan JC1 having the second axis described above as a rotation axis is provided near the base of the first jaw member J1. Then, the first wire C1 is wound around the first jaw capstan JC1 and the first motor capstan MC1, and a driving force generated by the first motor M1 is transmitted by the first wire C1, whereby the turning operation of the first jaw member J1 about the second axis is implemented. Furthermore, a second jaw capstan JC2 having the second axis described above as a rotation axis is provided near the base of the second jaw member J2. Then, the second wire C2 is wound around the second jaw capstan JC2 and the second motor capstan MC2, and a driving force generated by the second motor M2 is transmitted by the second wire C2, whereby the turning operation of the second jaw member J2 about the second axis is implemented.

Here, the first wire C1 and the second wire C2 are respectively wound around the first jaw capstan JC1 and the second jaw capstan JC2 from opposite directions. Specifically, when being pulled, the first wire C1 is wound around the first jaw capstan JC1 such that the first jaw member J1 turns in a direction approaching the second jaw member J2. Furthermore, when being pulled, the second wire C2 is wound around the second jaw capstan JC2 such that the second jaw member J2 turns in a direction approaching the first jaw member J1. Therefore, the opening or closing operation of the end effector can be performed by controlling pulling forces of the first wire C1 and the second wire C2 by the first motor M1 and the second motor M2 such that a difference in angle about the second axis between the first jaw member J1 and the second jaw member J2 changes. Furthermore, the end effector can be turned about the second axis by controlling the pulling forces of the first wire C1 and the second wire C2 by the first motor M1 and the second motor M2 such that the sum of angles about the second axis between the first jaw member J1 and the second jaw member J2 changes.

A spring SP is disposed between the first jaw member J1 and the second jaw member J2 such that a repulsive force constantly acts in an opening direction. As the spring SP, a torsion coil spring is preferably used. The spring SP has a natural length in which the repulsive force acts even at the maximum opening angle between the first jaw member J1 and the second jaw member J2. However, a method of mounting the spring SP is not particularly limited, and thus, the detailed description thereof will be omitted here.

The repulsive force acts between the first jaw member J1 and the second jaw member J2 due to a restoring force of the spring SP, and pre-tension constantly acts in the opening direction. Therefore, when the first jaw member J1 is pulled in a closing direction by the first motor M1 using one first wire C1 (in other words, only for the forward path) and the second jaw member J2 is pulled in the closing direction by the second motor M2 using one second wire C2 (in other words, only for the forward path), the first jaw member J1 and the second jaw member J2 can be closed. Furthermore, when the pulling by the first motor M1 and the second motor M2 is stopped, the first jaw member J1 and the second jaw member J2 are automatically opened by the restoring force of the spring SP. That is, since the operation of opening the first jaw member J1 and the second jaw member J2 is performed by an elastic force of the spring SP, the wire for the backward path to open the jaw members is unnecessary.

Referring to FIGS. 5 and 6, the first wire C1 attached to the first jaw capstan JC1 is pulled in a direction orthogonal to the second axis, but is redirected in a direction orthogonal to the first axis by a first idler pulley P1a having the first axis as a rotation axis. Moreover, a first adjacent idler pulley P1b, which is adjacent to the first idler pulley P1a and has a rotation axis parallel to the first axis, causes the first wire C1 to be inserted through the shaft 402 to be redirected in a longitudinal-axis direction of the shaft 402, and then, to be wound around the first motor capstan MC1 at the other end.

The first wire C1 is wound from a direction in which the distance from the first idler pulley P1a is minimized. Furthermore, the first wire C1 is wound such that the first idler pulley P1a and the first adjacent idler pulley P1b rotate in opposite directions when the first wire C1 is pulled. Then, when the first motor capstan MC1 is rotated by the first motor M1 to generate the pulling force of the first wire C1, a torque about the second axis can be applied to the first jaw member J1 to turn the first jaw member J1 in the direction approaching the second jaw member J2 (closing direction).

Furthermore, referring to FIGS. 5 and 6, the second wire C2 attached to the second jaw capstan JC2 is pulled in the direction orthogonal to the second axis, but is redirected in the direction orthogonal to the first axis by a second idler pulley P2a having the first axis as a rotation axis. Moreover, a second adjacent idler pulley P2b, which is adjacent to the second idler pulley P2a and has a rotation axis parallel to the first axis, causes the second wire C2 to be inserted through the shaft 402 to be redirected in a longitudinal-axis direction of the shaft 402, and then, to be wound around the second motor capstan MC2 at the other end.

The second wire C2 is wound from a direction in which the distance from the second idler pulley P2a is minimized. Furthermore, the second wire C2 is wound such that the second idler pulley P2a and the second adjacent idler pulley P2b rotate in opposite directions when the second wire C2 is pulled. Here, the direction in which the second wire C2 is wound around the second idler pulley P2a is opposite to the direction in which the first wire C1 is wound around the first idler pulley P1a. Then, when the second motor capstan MC2 is rotated by the second motor M2 to generate the pulling force of the second wire C2, a torque about the second axis can be applied to the second jaw member J2 to turn the second jaw member J2 in the direction approaching the first jaw member J1 (closing direction).

Due to the idler pulleys configured to redirect the first wire C1 and the second wire C2 in front of (that is, a vicinity of the first axis) the insertion into the shaft 402, the first wire C1 is redirected via the idler pulley P1b after passing through the shaft 402, and is wound around the first motor capstan MC1 at a terminal part. Similarly, after passing through the shaft 402, the second wire C2 is redirected via the idler pulley P2b and wound around the first motor capstan MC1 at a terminal part.

Furthermore, as illustrated in FIG. 4, the first wire C1 and the second wire C2 are wound around the first motor capstan MC1 and the second motor capstan MC2, respectively, and then, coupled via a tension spring TS1. Therefore, pre-tension generated by a restoring force of the tension spring TS1 is applied to the first wire C1 and the second wire C2.

Next, a specific operation method of the front end part 401 of the surgical instrument unit 400 will be described.

### Operation at First Axis:

The third wire including the wire C3a for the forward path and the wire C3b for the backward path is wound between the third motor capstan MC3 and the wrist capstan WC in a loop. When the third motor capstan MC3 is rotated by the third motor M3, a pulling force is generated on the third wire, and the wrist capstan WC can be rotated about the first axis. As a result, the wrist element WE and the end effector mounted on the wrist element WE can be turned about the first axis.

### Operation at Second Axis:

An average value of an angle about the second axis of the first jaw member J1 and an angle about the second axis of the second jaw member J2 is defined as an angle about the second axis of the end effector. When the first jaw capstan JC1 and the second jaw capstan JC2 are rotated in the same direction at the same speed, the turning operation of the end effector about the second axis is generated.

### Operation of End Effector:

The end effector includes the pair of opposing jaw members, that is, the first jaw member J1 and the second jaw member J2. The opening angle between the first jaw member J1 and the second jaw member J2 is set as an opening/closing angle of the end effector. When the first motor capstan MC1 and the second motor capstan MC2 are rotated in opposite directions at the same speed, the opening or closing operation of the end effector is generated.

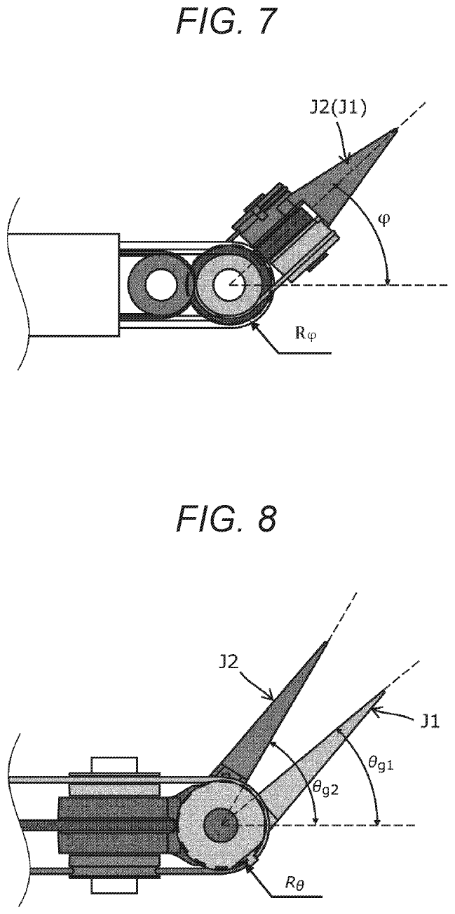

FIG. 7 illustrates an example of an operation of the wrist element WE about the first axis. Meanwhile, this drawing is a diagram of a surgical instrument unit front end part 101 as viewed from a direction parallel to the first axis. As illustrated in the drawing, a pulley radius of the wrist capstan WC is Ry, and a turning angle about the first axis of the wrist element WE is ψ.

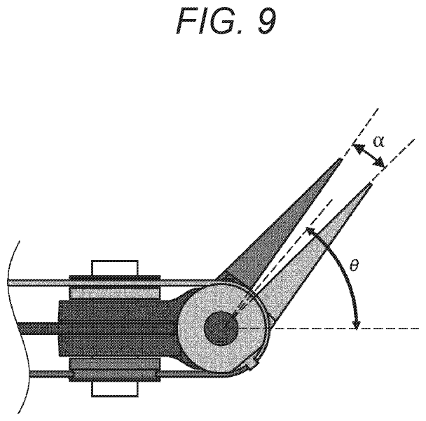

Furthermore, FIGS. 8 and 9 illustrate an example of an operation of the end effector about the second axis. Meanwhile, each of the drawings is a diagram of the surgical instrument unit front end part 101 as viewed from a direction parallel to the second axis. As illustrated in each of the drawings, a pulley radius of each of the first jaw capstan JC1 and the second jaw capstan JC2 is Re, a turning angle of the first jaw member J1 about the second axis is θ_{g1}, a turning angle of the second jaw member J2 about the second axis is θg₂, an opening angle of the end effector is α, and a turning angle of the end effector about the second axis is θ.

Furthermore, although not illustrated, a pulley radius of each of the first motor capstan MC1 and the second motor capstan MC2 is Rₘ₁₂, a pulley radius of the third motor capstan MC3 is Rₘ₃, a rotation angle of the first motor M1 is φₘ₁, a rotation angle of the second motor M2 is φₘ₂, and a rotation angle of the third motor M3 is φₘ₃.

Then, the turning angle ψ of the wrist element WE about the first axis, the turning angle θ of the end effector about the second axis, and the opening angle α of the end effector are expressed as the following Formulas (1) to (3), respectively.

$$\begin{matrix}[ Formula 1 ] & \\ ψ = \frac{{R}_{m 3}}{{R}_{ψ}} {ϕ}_{m 3} & ( 1 )\end{matrix}$$

$$\begin{matrix}[ Formula 2 ] & \\ θ = \frac{{θ}_{g 1} + {θ}_{g 2}}{2} & ( 2 )\end{matrix}$$

$$\begin{matrix}[ Formula 3 ] & \\ α = {θ}_{g 1} - {θ}_{g 2} & ( 3 )\end{matrix}$$

Furthermore, the turning angle θ_{g1} of the first jaw member J1 about the second axis and the turning angle θ_{g2} of the second jaw member J2 about the second axis are expressed by the following Formulas (4) and (5), respectively.

$$\begin{matrix}[ Formula 4 ] & \\ {θ}_{g 2} = \frac{{R}_{m 12}}{{R}_{θ}} {ϕ}_{m 2} + \frac{{R}_{ψ}}{{R}_{θ}} ψ & ( 4 )\end{matrix}$$

$$\begin{matrix}[ Formula 5 ] & \\ {θ}_{g 1} = - \frac{{R}_{m 12}}{{R}_{θ}} {ϕ}_{m 1} + \frac{{R}_{ψ}}{{R}_{θ}} ψ & ( 5 )\end{matrix}$$

As can be seen from the above Formulas (1) to (5), the turning angle φ of the wrist element WE about the first axis is not affected by the turning angles θ_{g1} and θ_{g12} of the first jaw member J1 and the second jaw member J2 about the second axis. On the other hand, the turning angle φ of the wrist element WE about the first axis affects the turning angles θ_{g1} and θ_{g12} of the first jaw member J1 and the second jaw member J2 about the second axis. Therefore, the target turning angle θ about the second axis and the target opening angle α of the end effector can be achieved by performing control so as to compensate for the influence of the turning angle φ of the wrist element WE about the first axis.

In short, the turning operation of the wrist element WE about the first axis can be controlled by controlling the rotation angle φₘ₃ of the third motor M3. Furthermore, it is possible to control the turning operation about the second axis and the opening and closing operations of the end effector by controlling the rotation angles φₘ₁, φₘ₂, and φₘ₃ of the first motor M1, the second motor M2, and the third motor M3.

### E. Surgical Instrument Device Using Drip-Proof Adapter

As described in the section D described above, the surgical instrument unit is configured to reduce the size of the front end in consideration of being inserted into a body cavity via a trocar and used and to transmit the driving force generated by the actuator in the drive unit arranged at the proximal end of the arm via the wire inserted into the hollow shaft to operate the surgical instrument.

Furthermore, since the surgical instrument is in direct contact with the body cavity, it is necessary to perform sterilization processing prior to use, but the drive unit does not have a structure capable of withstanding the sterilization processing. Therefore, the present disclosure adopts a structure in which the surgical instrument unit is attached to and detached from the drive unit via the adapter provided near the middle of the shaft. Therefore, the sterilization processing can be performed by detaching the surgical instrument unit from the drive unit via the adapter according to the present disclosure. Since the drive unit drives the surgical instrument unit using a translatory transmission mechanism such as the wire, the adapter according to the present disclosure is a connection device having a structure for that transmits the driving force in a translatory manner.

Moreover, the adapter according to the present disclosure has a drip-proof structure so as to maintain a state in which a clean region and an unclean region are separated when the surgical instrument unit subjected to the sterilization processing is attached or while the surgical instrument such as the jaw member at the front end is driven. To summarize the above, the adapter according to the present disclosure is the connection device having the drip-proof structure and the translatory transmission structure.

### E-1. Overall Configuration of Surgical Instrument Device

FIG. 10 illustrates a configuration example of a surgical instrument device 1000 to which the present disclosure is applied. The surgical instrument device 1000 includes a surgical instrument unit 1001, an adapter 1002, and a drive unit 1003 in order from a front end (distal end). In the example illustrated in FIG. 10, the surgical instrument unit 1001 includes an end effector including a pair of jaw members and the like and a hollow shaft that supports the end effector, and a translatory transmission mechanism (not illustrated) such as a wire is inserted into the shaft. Then, the surgical instrument unit 1001 is connected to the drive unit 1003 via the adapter 1002 at a base part (or proximal end) of the shaft. Furthermore, FIG. 11 illustrates an exploded view of the surgical instrument device 1000. In the example illustrated in FIG. 11, the surgical instrument unit 1001, the adapter 1002, and the drive unit 1003 are drawn separately in a longitudinal-axis direction of the shaft.

The adapter 1002 has a translatory transmission structure to transmit a driving force generated by the drive unit 1003 to the surgical instrument unit 1001, and a drip-proof structure configured to maintain a state in which a clean region on the surgical instrument unit 1001 side and an unclean region on the drive unit 1003 side are separated. Furthermore, a drape 1004 is used to cover the unclean region on the drive unit 1003 side (or isolate the clean region on the surgical instrument unit 1001 side). It is assumed that the adapter 1002 and the drape 1004 are integrated, or there is no gap between the adapter 1002 and the drape 1004 so that a substance in the unclean region does not permeate into and contaminate the clean region. The surgical instrument unit 1001 may be reusable a plurality of times by sterilization processing. Furthermore, the adapter 1002 and the drape 1004 may be disposable after one surgical operation.

### E-2. Translatory Transmission Structure of Adapter

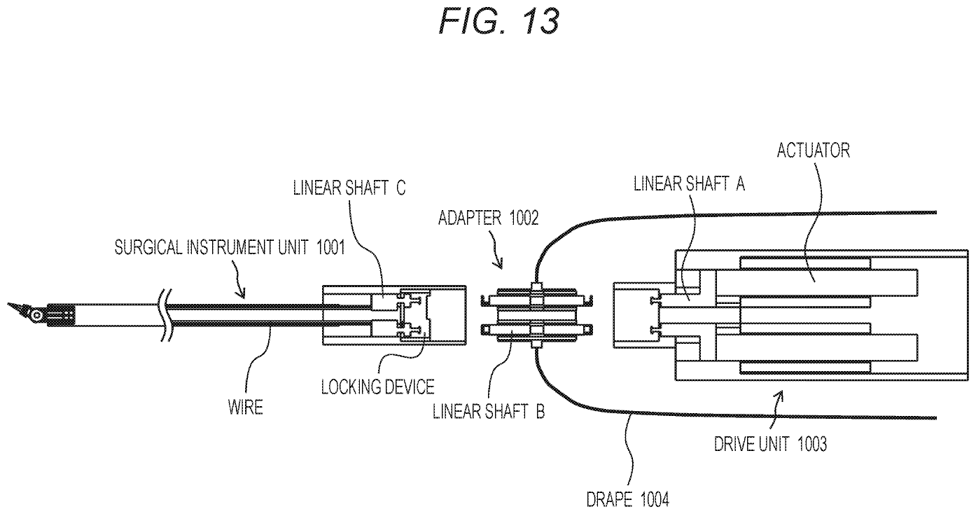

FIG. 12 illustrates a cross section of the surgical instrument device 1000 in a state in which the surgical instrument unit 1001 is attached to the drive unit 1003 via the adapter 1002. Furthermore, FIG. 13 illustrates a cross section of the surgical instrument device 1000 in a state in which the surgical instrument unit 1001, the adapter 1002, and the drive unit 1003 are separated. Meanwhile, the cross sections are taken along a plane parallel to a longitudinal axis of the shaft. The translatory transmission structure of the adapter 1002 will be described with reference to FIGS. 12 and 13. Note that the configuration example in which the four wires (C1, C2, C3a, and C3b) are inserted into the shaft to drive the surgical instrument has been described in the section D described above, but the following description will be given assuming that the number of wires is two in order to prevent the complication of the drawing. It is only required to add a similar translatory transmission structure according to the number of wires to be actually used.

For each wire, a linear shaft A operated in a longitudinal direction by a corresponding actuator in the drive unit 1003, a linear shaft B in the adapter 1002 corresponding to the linear shaft A, and a linear shaft C in the surgical instrument unit 1001 corresponding to the linear shaft B are disposed. A front end of the linear shaft C is coupled with an end part of a corresponding wire. The surgical instrument unit 1001 includes a locking device at a junction with the adapter 1002 at a rear end (or proximal end). The locking device has a function of fixing a position of the linear shaft C and releasing the fixing. Details of the locking device will be described later in detail in the section E-4.

As illustrated in FIG. 12, a projection part at a front end of the linear shaft A and a receiving part at a rear end of the linear shaft B are coupled when the adapter 1002 is attached to the drive unit 1003, and a receiving part at a front end of the linear shaft B and a projection part at a rear end of the linear shaft C are coupled when the surgical instrument unit 1001 is attached to the adapter 1002. Therefore, the linear shafts A to C are integrally driven in the longitudinal-axis direction of the shaft in a state in which the surgical instrument unit 1001 is attached to the drive unit 1003 via the adapter 1002. When the actuator in the drive unit 1003 is driven, a pulling force can be transmitted to the corresponding wire through the linear shafts A to C in a translatory manner. Note that details of an attachment and detachment procedure of the surgical instrument unit 1001, the adapter 1002, and the drive unit 1003 will be described later in detail.

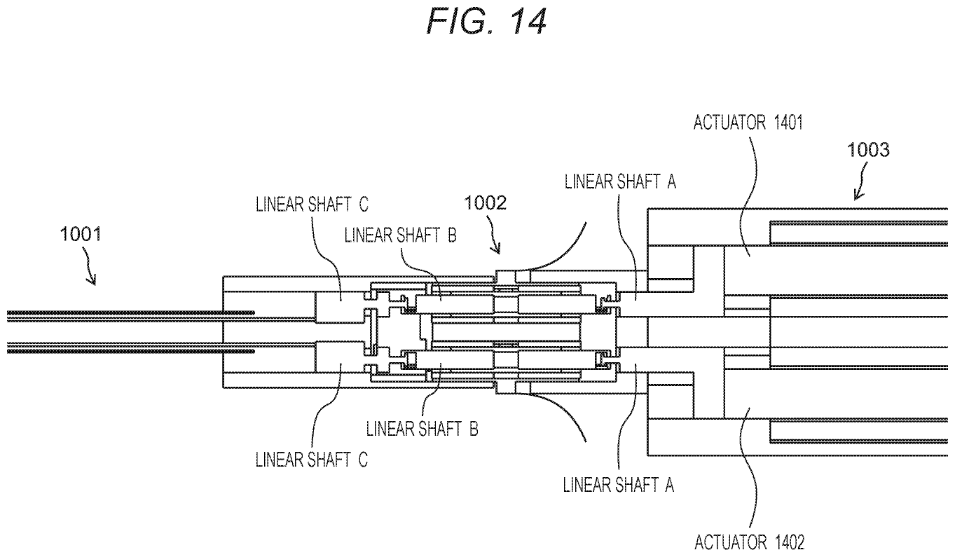

FIG. 14 illustrates an enlarged cross section of a vicinity of the adapter 1002 immediately after attachment, that is, in a state in which the surgical instrument unit 1001 and the drive unit 1003 are attached. Two actuators 1401 and 1402 are illustrated on the illustrated cross section. The linear shafts A are attached to output shafts of the actuators 1401 and 1402, respectively. Then, the front end of the linear shaft A and the rear end of the linear shaft B are coupled when the adapter 1002 is attached to the drive unit 1003, and the front end of the linear shaft B and the rear end of the linear shaft C are coupled when the surgical instrument unit 1001 is attached to the adapter 1002. A method of coupling the linear shaft A and the linear shaft B and a method of coupling the linear shaft B and the linear shaft C will be described in detail in the section F below.

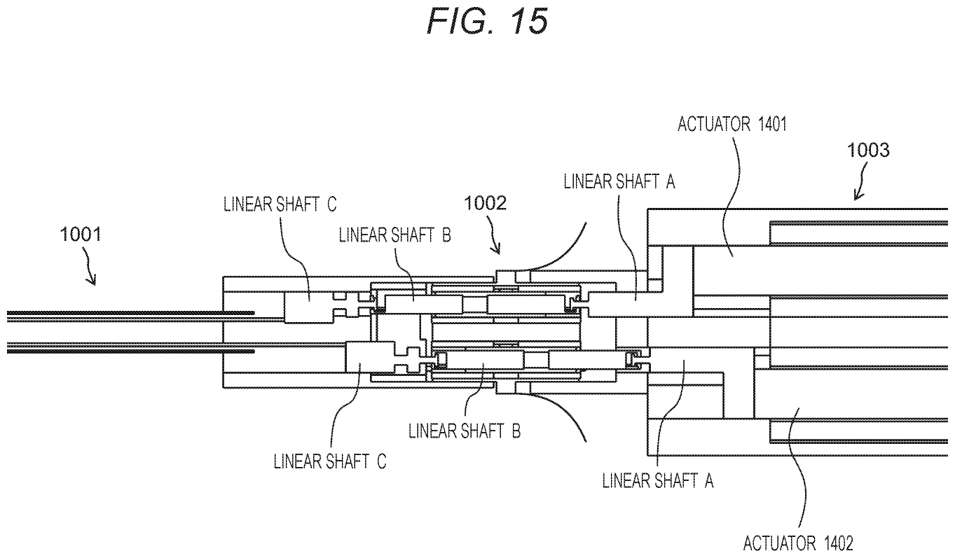

FIG. 15 illustrates an enlarged cross section of the vicinity of the adapter 1002 during operation, that is, when the actuators 1401 and 1402 in the drive unit 1003 are driven. In the example illustrated in FIG. 15, the actuator 1401 on the upper side of the paper advances toward a front end (or distal end), and this driving force is transmitted in a translatory manner to the wires on the surgical instrument unit 1001 side via the linear shafts A to C. Furthermore, the actuator 1402 on the lower side of the paper retreats toward the rear side (or a proximal end), and this driving force is transmitted in a translatory manner to the wires on the surgical instrument unit 1001 side via the linear shafts A to C.

Operations and roles of parts of each of the drive unit 1003, the adapter 1002, and the surgical instrument unit 1001 at the time of driving the surgical instrument device 1000 will be described with reference to FIGS. 12 to 15.

### Regarding Drive Unit:

The drive unit 1003 can drive the actuator to move the linear shaft A back and forth along the longitudinal axis. The forward movement corresponds to advancement of the linear shaft A toward the front end (or distal end), and the backward movement corresponds to retreat of the linear shaft A toward an end (or proximal end). The actuator preferably includes a sensor capable of detecting a current position of the linear shaft A, such as a translatory encoder.

The drive unit 1003 is coupled with a front end of the arm 210 of the medical arm device 110 (see FIGS. 1 and 2) and drives the actuator to operate on the basis of a control signal from the control device 120 such as a master, or is coupled with a front end of the surgical manipulation device 300 (see FIG. 3) held and manipulated by a hand of a surgeon and drives the actuator to operate on the basis of the manipulation performed by the surgeon on the handle part 310, for example.

### Adapter:

The adapter 1002 includes the linear shaft B as a translatory transmission part configured to transmit a driving force generated by the drive unit 1003, in other words, motion of the linear shaft A in the longitudinal-axis direction, to the surgical instrument unit 1001. The linear shaft B is coupled with the front end of the linear shaft A at a rear end part, and is coupled with a rear end part of the linear shaft C at a front end part. Therefore, when the actuator is driven on the drive unit 1003 side and the linear shaft A moves back and forth along the longitudinal-axis direction, the linear shaft B follows this movement and moves back and forth in the longitudinal-axis direction, and as a result, the linear shaft C of the surgical instrument unit 1001 also moves back and forth in the longitudinal-axis direction, whereby the driving force of the actuator in the drive unit 1003 is transmitted in a translatory manner to the surgical instrument unit 1001 via the adapter 1002.

The surgical instrument unit 1001 is the clean region subjected to the sterilization processing prior to use, whereas the drive unit 1003 is the unclean region that is hardly subjected to the sterilization processing, and the adapter 1002 has a role of separating the clean region and the unclean region. The adapter 1002 has a structure in which the clean region and the unclean region are separated so as not to be mixed when the linear shaft B as the translatory transmission part moves back and forth in the longitudinal-axis direction. The separation structure of the adapter 1002 will be described in detail in the next section E-3.

### Surgical Instrument Unit:

The front end of the linear shaft C is coupled with the wire (see the section D described above) that pulls the end effector, such as the jaw member. Therefore, the linear shaft C moves back and forth in the longitudinal-axis direction by the linear shaft B as the translatory transmission part of the adapter 1002, and pulls the wire, so that the operations of the end effector such as the opening and closing operations of the jaw member and the turning operation of the wrist can be implemented.

### E-3. Drip-Proof Structure of Adapter

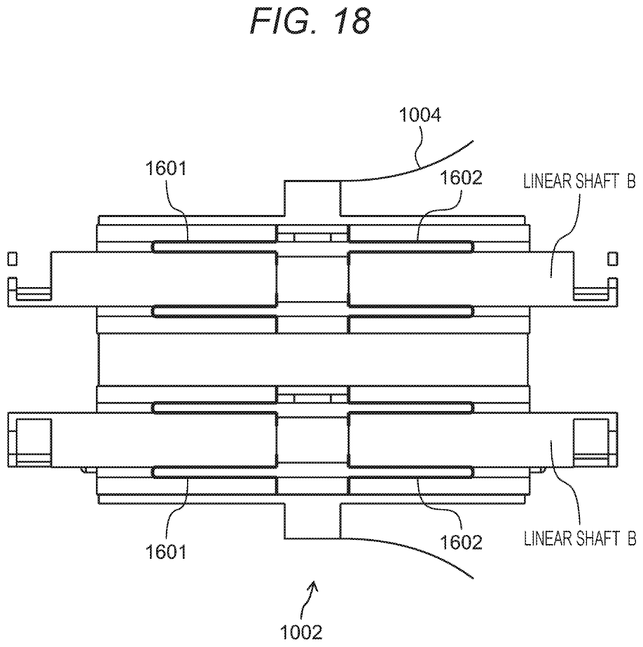

FIGS. 16 and 17 are perspective cross-sectional views of the adapter 1002 taken along a plane parallel to the longitudinal-axis direction. FIG. 16 illustrates a state in which each of the linear shafts B is at an initial position immediately after attachment, and FIG. 17 illustrates a state in which each of the linear shafts B has moved (a state in which the upper linear shaft B advances toward the front end (distal end side) and the lower linear shaft B retreats to the rear side (proximal end side)).

A main body of the adapter 1002 has a substantially cylindrical shape with the longitudinal-axis direction as a height direction, and through-holes 1611 allowing insertion of the number of the linear shafts B corresponding to the number of translatory transmission mechanisms are formed in the longitudinal-axis direction. Meanwhile, FIGS. 16 and 17 illustrates only two through-holes 1611 through which the linear shaft B and the linear shaft B are inserted in order to prevent the complication of the drawing. Furthermore, a rib 1612 is formed in an outer circumferential direction near the center of the cylinder of the main body of the adapter 1002. As can be seen from FIGS. 12 to 15, the cylinder of the main body of the adapter 1002 is received by the drive unit 1003 on one side and received by the surgical instrument unit 1001 on the other side. The rib 1612 corresponds to a position where the drive unit 1003 and the surgical instrument unit 1002 inserted from both sides of the cylinder are locked. Springs (not illustrated in FIGS. 16 and 17) applying reaction forces to the drive unit 1003 and the surgical instrument unit 1002 that have abutted thereon are attached to both side surfaces of the rib 1612.

A pair of tubular elastic bodies (shield rubbers in the present embodiment) 1601 and 1602 is attached to each of the linear shafts B. In the upper part of FIG. 16, appearances and cross sections of the shield rubbers 1601 and 1602 are enlarged. Each of the shield rubbers 1601 and 1602 includes a shield rubber having a two-fold structure of a flange type (or whose appearance is similar to a shape of a top hat). As illustrated in the lower part of FIG. 16, each of the shield rubber elastic bodies 1601 and 1602 follows an outer peripheral surface of the linear shaft B and then follows an inner peripheral surface of the through-hole 1611 when being folded outward. With the movement of the linear shaft B in the longitudinal-axis direction, each of the shield rubbers 1601 and 1602 can be deformed by rolling (rolled) while changing a fold length smoothly with low friction and without oil supply.

As illustrated in FIG. 16, the shield rubber 1601 and the shield rubber 1602 are symmetrically arranged such that end parts corresponding to brims of the respective “top hats” face each other. As both ends of the shield rubber 1601 and the shield rubber 1602 are connected with the linear shaft B, a structure having a sealed air chamber is obtained. Therefore, the clean region on the front end side (or distal end side) and the unclean region on the rear end side (or proximal end side) of the linear shaft B are completely separated.

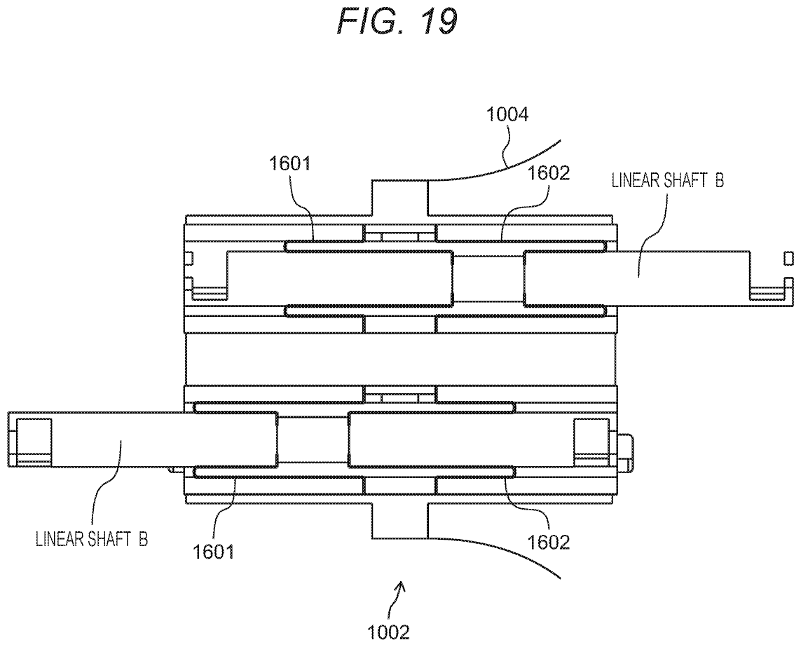

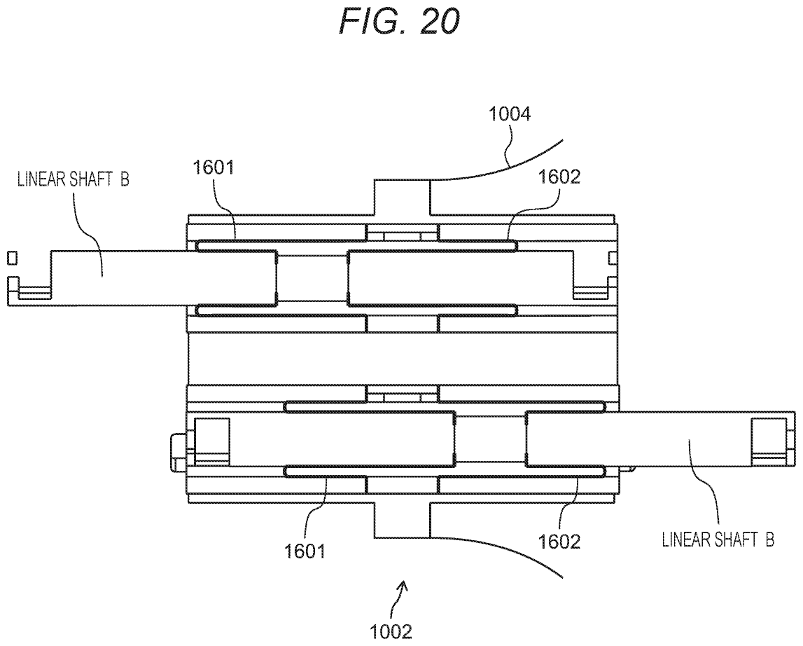

FIG. 18 illustrates a state in which the two linear shafts B are in initial positions immediately after attachment, FIG. 19 illustrates a state in which the upper linear shaft B retreats to the rear side (proximal end side) and the lower linear shaft B advances to the front end (distal end side), and FIG. 20 illustrates a cross section of the adapter 1002 in a state in which the upper linear shaft B advances to the front end (distal end side) and the lower linear shaft B retreats to the rear side (proximal end side). Referring to FIGS. 18 to 20, in any state, the clean region on the front end side (or distal end side) and the unclean region on the rear end side (or proximal end side) of the linear shaft B are completely separated via the air chamber defined by the pair of shield rubbers 1601 and 1602, and the clean region is not contaminated by mixing of the regions.

The volume of the air chamber can be maintained constant regardless of the position of the linear shaft B in the longitudinal-axis direction within a predetermined movable range. As illustrated in FIGS. 18 to 20, the shield rubbers 1601 and 1602 are smoothly deformed by rolling (rolled) to move positions of fold lines in the longitudinal-axis direction accompanying the translatory movement in which the linear shaft B advances to the front end (distal end side) or retreats to the rear side (proximal end side), thereby changing heights of crowns of the “top hats” according to the current position of the linear shaft B. The reaction forces such as restoring forces are not generated in the shield rubbers 1601 and 1602 due to such rolling deformation. Since the respective shield rubbers 1601 and 1602 are smoothly deformed by rolling, the linear shaft B can be supported with low friction and without oil supply.

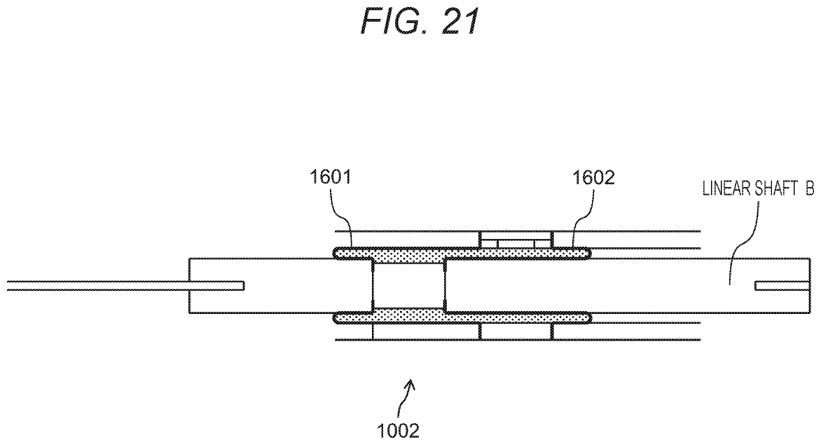

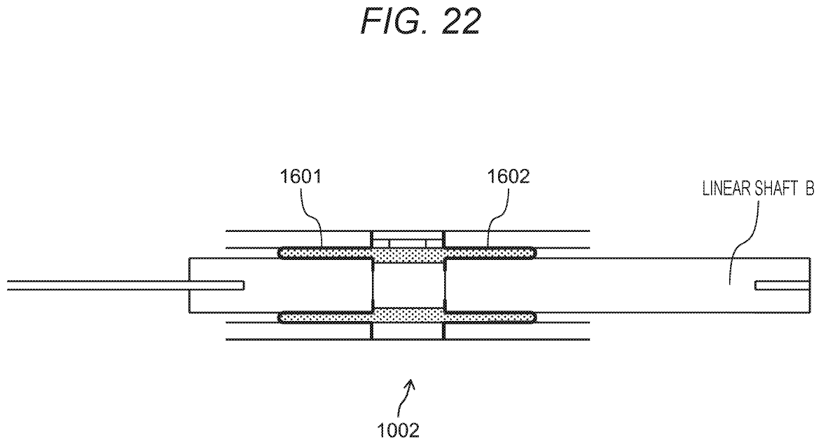

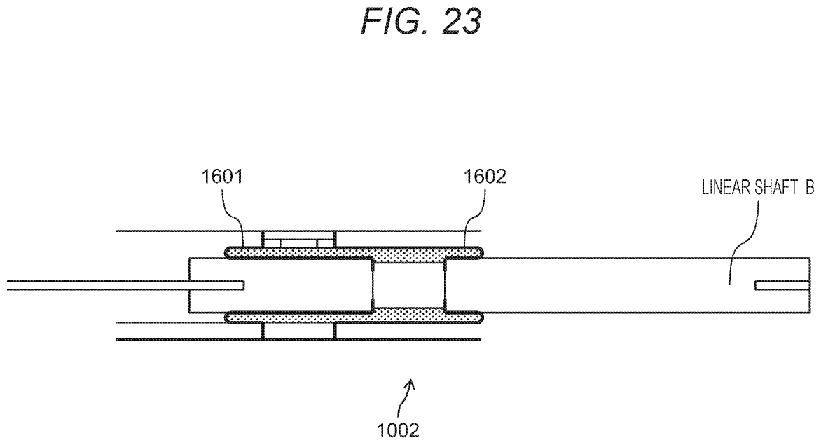

FIGS. 21 to 23 illustrate changes of the air chamber accompanying the movement of the linear shaft B in the longitudinal-axis direction. Meanwhile, the inside of the air chamber is filled with dots in each drawing. FIG. 21 illustrates a state in which the linear shaft B has advanced to the front end (distal end side), FIG. 22 illustrates a state in which the linear shaft B is at the initial position, and FIG. 23 illustrates a state in which the linear shaft B has retreated to the rear side (proximal end side).

A preload is applied to the air chamber to the extent that the shield rubbers 1601 and 1602 strain. As illustrated in FIGS. 21 to 23, the volume of the air chamber is always constant while the linear shaft B moves in the longitudinal-axis direction. Therefore, resistance due to pressurization and depressurization of the air chamber is not generated when the linear shaft B moves in the longitudinal-axis direction. Furthermore, there is no sliding part, such as a sliding bearing, between the linear shaft B and the through-hole 1611, and the surgical instrument unit 1001 side and the drive unit 1003 side are completely separated by the air chamber formed in the gap between the pair of shield rubbers 1601 and 1602, and thus, a complete drip-proof structure can be implemented. Furthermore, a tight fitting tolerance is not required in order to insert the linear shaft B through the through-hole 1611, and thus, the adapter 1002 can be manufactured at low cost.

As can also be seen in FIGS. 21 to 23, the drip-proof structure in the adapter 1002 can be achieved using the pair of shield rubbers 1601 and 1602 facing each other. As illustrated in the upper part of FIG. 16, the shield rubbers 1601 and 1602 having the two-fold structure of the flange type have a simple shape, so that size reduction is easy, and cost can be reduced by mass production. The shield rubbers 1601 and 1602 can also be referred to as “rolling diaphragms”.

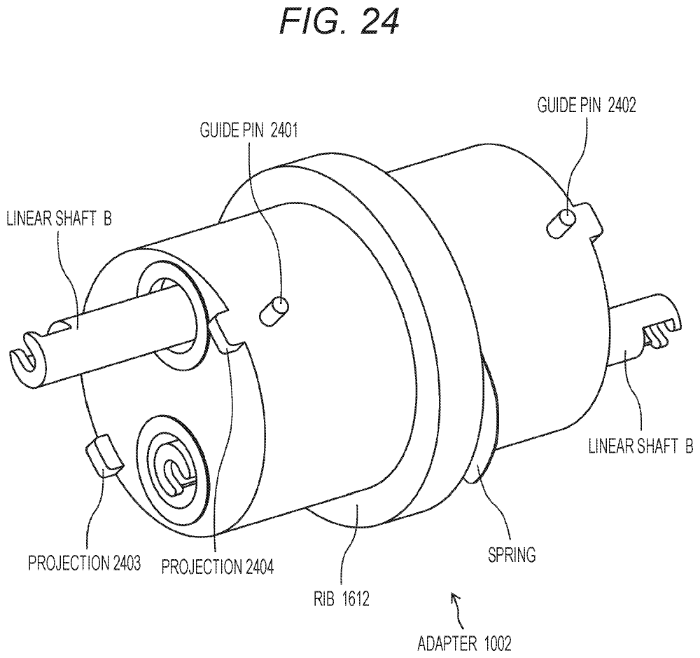

FIG. 24 illustrates an example of an external configuration of the adapter 1002. The adapter 1002 has a substantially cylindrical shape with a height in the longitudinal-axis direction, and each end surface of the cylinder is joined to each of the surgical instrument unit 1001 and the drive unit 1003. Furthermore, the linear shafts B forming the translatory transmission mechanisms protrude and retract from the respective end surfaces of the adapter 1002.

One guide pin 2401 and one guide pin 2402 protrude from a side surface of the adapter 1002, respectively, on the front end side (or distal end side) and the rear end side (or proximal end side). Each of the guide pins 2401 and 2402 has a role of guiding a manipulation direction at the time of attachment to the surgical instrument unit 1001 and the drive unit 1003, and details thereof will be described later.

Furthermore, on the end surface on the front end side (or distal end side) of the adapter 1002, two protrusions 2403 and 2404 are provided to protrude at positions different by 180 degrees about the longitudinal axis. These protrusions 2403 and 2404 regulate a rotation range of the locking device when the surgical instrument unit 1001 has been attached to the adapter 1002, and details thereof will be described in the next section E-4.

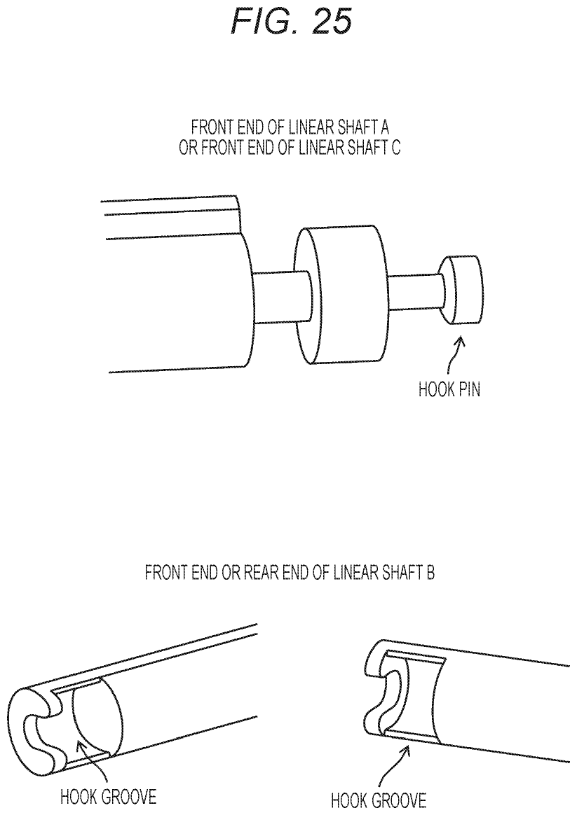

FIG. 25 illustrates a shape of a rear end portion of the linear shaft C and a shape of a front end portion of the linear shaft B. Although not illustrated, a shape of a front end portion of the linear shaft A is the same as the shape of the rear end portion of the linear shaft C, and furthermore, a shape of a rear end portion of the linear shaft B is also the same. The linear shaft C has a projection (hereinafter, also referred to as a “hook pin”) at the rear end. Similarly, the linear shaft A has a hook pin at the front end. The hook pins of the linear shaft A and the linear shaft C are formed to have substantially the same shape and the same dimension. On the other hand, the front end portion and the rear end portion of the linear shaft B each have a recessed part (hereinafter, also referred to as a “hook groove”) as illustrated in FIG. 25. The hook grooves at both ends of the linear shaft B has openings that receive the hook pins of the linear shaft A and the linear shaft C, and U-shaped notches that lock the hook pins entering from the openings. A scheme for coupling and separating the hook pin and the hook groove between the linear shaft A and the linear shaft B and a scheme for coupling and separating the hook pin and the hook groove between the linear shaft C and the linear shaft B will be described in the section F below together with an attachment and detachment procedure between the adapter 1002 and the drive unit 1003 and an attachment and detachment procedure between the surgical instrument unit 1001 and the adapter 1002.

### E-4. Locking Device of Surgical Instrument Unit

As described above, inside the surgical instrument unit 1001, the linear shafts C respectively corresponding to the wires that drive the front end (jaw member or the like) are arranged along the longitudinal-axis direction, and each of the linear shafts C is coupled with an end part of a corresponding wire. The surgical instrument unit 1001 includes the locking device that fixes (locks) a position of the linear shaft C and releases (unlocks) the fixation.

When the connection of the surgical instrument unit 1001 to the adapter 1002 is not completed, the locking device mainly play a role of fixing the linear shaft C (or restricting the movement of the linear shaft C) in a lock-on state and a role of preventing the adapter 1002 from being detached from the surgical instrument unit 1001 in a lock-off state. In this section, the locking device will be described in detail.

FIG. 26 illustrates an appearance (a front surface, a side surface, a rear surface, and a three-dimensional view) of a locking device 2600. The locking device 2600 is attached to the inside of a tubular receiving part that receives the adapter 1002 and provided at a rear end (or on a proximal end side) of the surgical instrument unit 1001 so as to be rotatable about a longitudinal axis along an inner periphery. The locking device 2600 has a shape like a cup without a bottom, the shape having an opening on a bottom surface 2601. The bottom surface 2601 has a locking claw 2602 having a step and a release part 2603 having an expanded opening radius. Furthermore, a manipulating element 2604 configured to manipulate locking and unlocking protrudes from an outer periphery of the locking device 2600. A user such as a surgeon can manipulate locking and unlocking by rotating the locking device 2600 about the longitudinal axis using the manipulating element 2604 to change rotational positions of the locking claw 2602 and the release part 2603.

Furthermore, the locking device 2600 has an uneven shape formed at an end part corresponding to an edge of the cup shape, and has two recesses 2605 and 2606 at positions different by 180 degrees about the longitudinal axis. These recesses 2605 and 2606 have a role of determining a rotation range of the locking device 2600 about the longitudinal axis by allowing insertion of the two protrusions 2403 and 2404 protruding from the end surface on the front end side (or distal end side) of the adapter 1002 at the time of attachment to the adapter 1002.

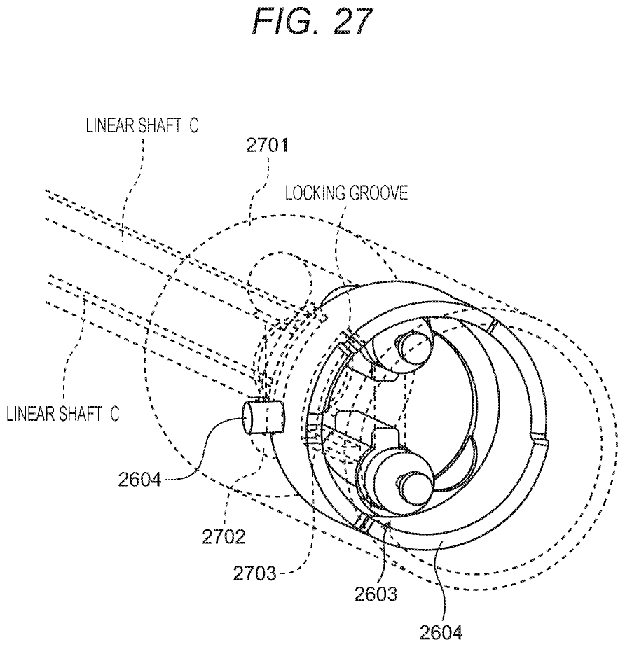

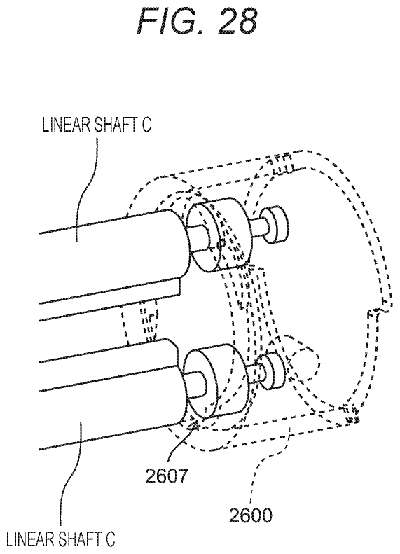

FIGS. 27 and 28 illustrate an external configuration of the locking device 2600 in the lock-off state together with the linear shaft C whose movement is to be restricted. Meanwhile, FIG. 27 illustrates a state in which the locking device 2600 is viewed from the rear end (or the proximal end side) of the surgical instrument unit 1001, and FIG. 28 illustrates a state in which the locking device 2600 is viewed from the front end (or the distal end side) of the surgical instrument unit 1001. In a tubular receiving part 2701 at the rear end of the surgical instrument unit 1001, a linear groove 2702 is formed along a circumferential direction. The manipulating element 2604 protruding from the outer periphery of the locking device 2600 is exposed from the receiving part 2701 to the outside through the groove 2702. The user such as the surgeon can rotate the locking device 2600 about the longitudinal axis using the manipulating element 2604. At a rotational position of the locking device 2600 illustrated in FIGS. 27 and 28, the rear ends of the two linear shafts C are located at the release part 2603 having the expanded opening radius, and thus, the movement of the linear shaft C is not restricted. Therefore, the rotational position of the locking device 2600 illustrated in FIGS. 27 and 28 can be referred to as a lock-off state or an unlocked position.

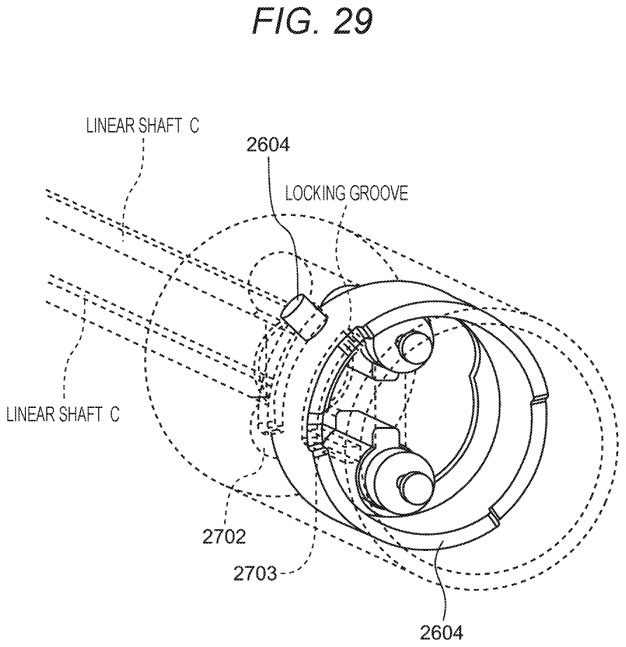

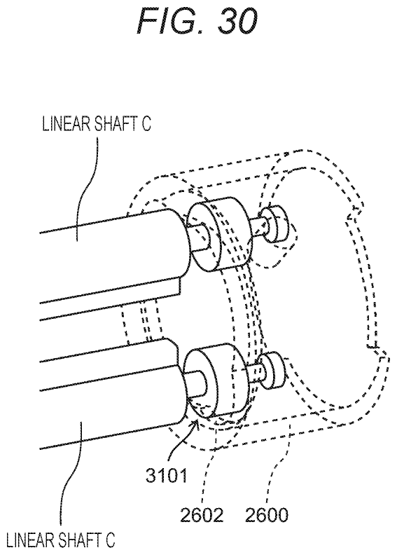

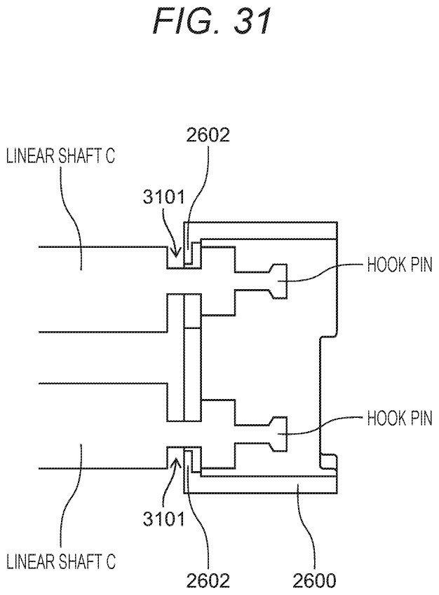

FIGS. 29 and 30 illustrate an external configuration of the locking device 2600 in the lock-on state. Meanwhile, FIG. 29 illustrates a state in which the locking device 2600 is viewed from the rear end (or the proximal end side) of the surgical instrument unit 1001, and FIG. 30 illustrates a state in which the locking device 2600 is viewed from the front end (or the distal end side) of the surgical instrument unit 1001. Furthermore, FIG. 31 illustrates a cross section of the locking device 2600 in the lock-on state. As described above, the user such as the surgeon can rotate the locking device 2600 about the longitudinal axis using the manipulating element 2604. At a rotational position of the locking device 2600 illustrated in FIGS. 29 and 30, the rear ends of the two linear shafts C abut on the locking claw 2602 having a step. Referring to FIG. 31, a constricted part 3101 is formed in front of the hook pin at the rear end of the linear shaft C. Then, at the rotational position of the locking device 2600 illustrated in FIGS. 29 and 30, the locking claw 2602 locks the constricted part 3101 to fix the linear shaft C (or restrict the movement of the linear shaft C). Therefore, the rotational position of the locking device 2600 illustrated in FIGS. 29 and 30 can be referred to as a lock-on state or a locked position. When the linear shaft C is fixed, the wire coupled with the linear shaft C is not movable in the longitudinal-axis direction either, and thus, the operation of the end effector such as the jaw member is restricted.

Referring again to FIG. 26, the uneven shape is formed at the end part corresponding to the edge of the cup shape of the locking device 2600, and the two recesses 2605 and 2606 are provided at positions different by 180 degrees about the longitudinal axis. On the other hand, referring again to FIG. 24, the two protrusions 2403 and 2404 are provided on the end surface on the front end side (or distal end side) of the adapter 1002 to protrude at positions different by 180 degrees about the longitudinal axis.

The surgical instrument unit 1001 can be attached to the adapter 1002 at a relative rotational position in which the two protrusions 2403 and 2404 of the adapter 1002 are respectively accommodated in the two recesses 2605 and 2606 of the locking device 2600. As a lock layer 2600 is rotated about the longitudinal axis, the two protrusions 2403 and 2404 on the adapter 1002 side collide with terminal ends of the recesses 2605 and 2606 of the locking device 2600, respectively, whereby the rotation is regulated. That is, the locking device 2600 can be rotated about the longitudinal axis within a range in which the protrusions 2403 and 2404 fit in the recesses 2605 and 2606, respectively.

Next, a relationship between a manipulation of attaching the surgical instrument unit 1001 to the adapter 1002 and switching locking and unlocking of the locking device 2600 will be described.

Referring to FIGS. 27 and 29, a linear L-shaped groove 2703, which has an L shape and advances in a circumferential direction by being bent at a right angle after advancing in the longitudinal-axis direction from an edge of a rear end, is formed in the tubular receiving part 2701 of the surgical instrument unit 1001. The L-shaped groove 2703 has a locking groove bent in the longitudinal-axis direction at a toe of an L-shaped foot. Furthermore, referring again to FIG. 24, the guide pin 2401 protrudes from the side surface on the front end side (or distal end side) of the adapter 1002.

The surgical instrument unit 1001 is first inserted into the adapter 1002 in the longitudinal-axis direction such that the guide pin 2401 follows the L shape of the L-shaped groove 2703 with the guide pin 2401 on the adapter 1002 side being aligned with an entrance of the L-shaped groove 2703 of the receiving part 2701 on the surgical instrument unit 1001 side, and then, the surgical instrument unit 1001 is rotated about the longitudinal axis by about 45 degrees when reaching a bent portion of the L shape. Then, the surgical instrument unit 1001 can be attached to the adapter 1002 by pushing the guide pin 2401 into the locking groove at the innermost part of the L-shaped groove 2703 using the reaction force of the spring (described above) attached to the side surface of the rib 1612 finally when reaching the toe of the L-shaped foot of the L-shaped groove 2703.

When the locking device 2600 is set in the lock-off state in a state in which the guide pin 2401 of the adapter 1002 is fitted in the L-shaped groove 2703 having the L shape, the restriction on the movement of the linear shaft C is released, and the guide pin 2401 of the adapter 1002 is locked to restrict the detachment of the surgical instrument unit 1001.

Meanwhile, an attachment and detachment procedure of the surgical instrument unit 1001 and the adapter 1002 will be described in detail in the next section F.

Note that the locking device 2600 is pressed by pre-tension of the wire in the surgical instrument unit 1001 when the locking device 2600 is locked in a state in which the surgical instrument unit 1001 is not attached to the adapter 1002. Therefore, there is a low risk that the locking device 2600 is unexpectedly detached.

### F. Attachment and Detachment Procedure

In a case where the sterilized surgical instrument unit 1001 is used, attachment is performed in the order of first attaching the adapter 1002 to the drive unit 1003 and then attaching the surgical instrument unit 1001 to the adapter 1002. On the other hand, in a case where the surgical instrument unit 1001 after use is replaced, detachment is performed in the order of first detaching the surgical instrument unit 1001 from the adapter 1002 and then detaching the adapter 1002 from the drive unit 1003.

### F-1. Attachment of Adapter to Drive Unit

A procedure for attaching the adapter 1002 to the drive unit 1003 will be described with reference to FIGS. 32 to 35.

First, the actuator is driven in the drive unit 1003 to stop all the linear shafts A at attachment and detachment positions (see FIG. 32). The attachment and detachment position of the linear shaft A is a reference position in the longitudinal-axis direction of the linear shaft A for appropriately coupling corresponding end parts of the linear shaft A and linear shaft B when the adapter 1002 is attached to the drive unit 1003, and is defined in advance.

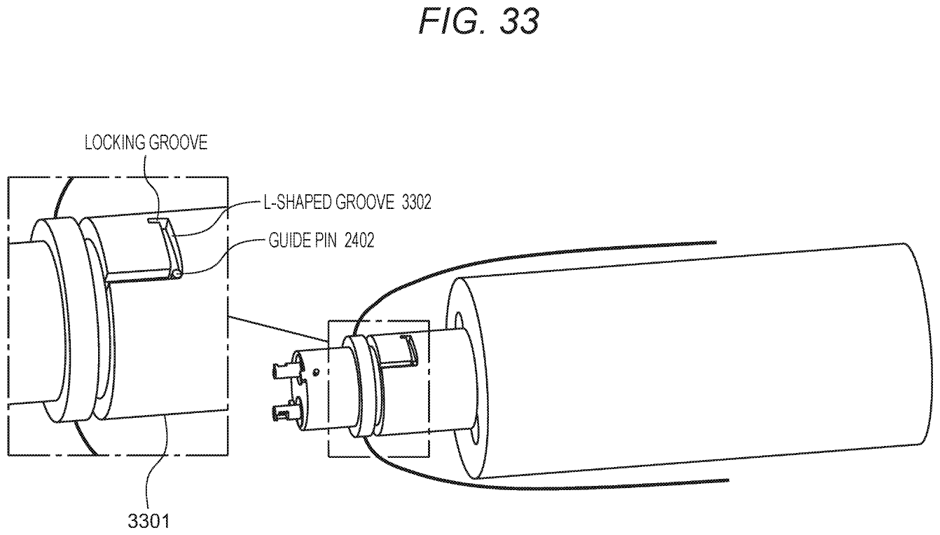

As illustrated in an enlarged manner on the left side of FIG. 33, the drive unit 1003 includes a tubular receiving part 3301 that receives the adapter 1002 at a front end part. Similarly to the receiving part 2701 of the surgical instrument unit 1001 (described above), a linear L-shaped groove 3302, which has an L shape and advances in a circumferential direction by being bent at a right angle after advancing in the longitudinal-axis direction from an edge of a rear end, is formed in the receiving part 3301. The L-shaped groove 3302 has a locking groove bent in the longitudinal-axis direction at a toe of an L-shaped foot. Furthermore, referring again to FIG. 24, the guide pin 2402 protrudes from the side surface on the rear end side (or proximal end side) of the adapter 1002.

The guide pin 2402 is inserted into the L-shaped groove 3302 with the guide pin 2402 on the adapter 1002 side being aligned with an entrance of the L-shaped groove 3302 having the L shape of the receiving part 3302 on the drive unit 1003 side, and the adapter 1002 is inserted into the receiving part 3301 in the longitudinal-axis direction such that the guide pin 2402 follows the L shape of the L-shaped groove 3302 (see FIG. 33).

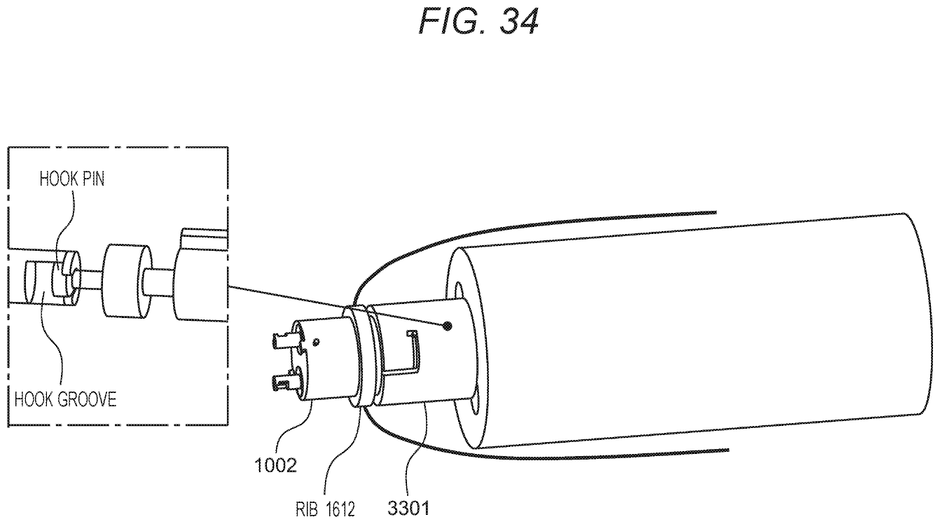

When the guide pin 2402 reaches a bent portion of the L shape of the L-shaped groove 3302, the adapter 1002 is then rotated about the longitudinal axis by about 45 degrees with respect to the drive unit 1003 (see FIG. 34). A front end edge of the receiving part 3301 on the drive unit 1003 side just abuts on the rib 1612 of the adapter 1002 at a position where the guide pin 2402 reaches the bent portion of the L shape of the L-shaped groove 3302. As described with reference to FIG. 25, the hook pin is formed at the front end of the linear shaft A, and the hook groove is formed at the rear end of the linear shaft B. The hook groove has the opening that receives the hook pin and the U-shaped notch that locks the hook pin entering from the opening. When the guide pin 2402 reaches the bent portion of the L shape of the L-shaped groove 3302, the hook pin and the hook groove are positioned to just overlap each other, and the opening of the hook groove faces the hook pin. Then, the hook pin at the front end of the linear shaft A is accommodated in the opening of the hook groove of the linear shaft B and is hooked by the U-shaped notch (see the left side of FIG. 34) in the course of rotation of the adapter 1002 about the longitudinal axis by about 45 degrees with respect to the drive unit 1003, whereby the linear shaft B is coupled with the linear shaft A. When the adapter 1002 is attached to the receiving part 3301 on the drive unit 1003 side such that the guide pin 2402 is set along the L-shaped groove 3302, the hook pin at the front end of the linear shaft A can be guided to follow a path of being hooked by the notch of the hook groove at the rear end of the linear shaft B.

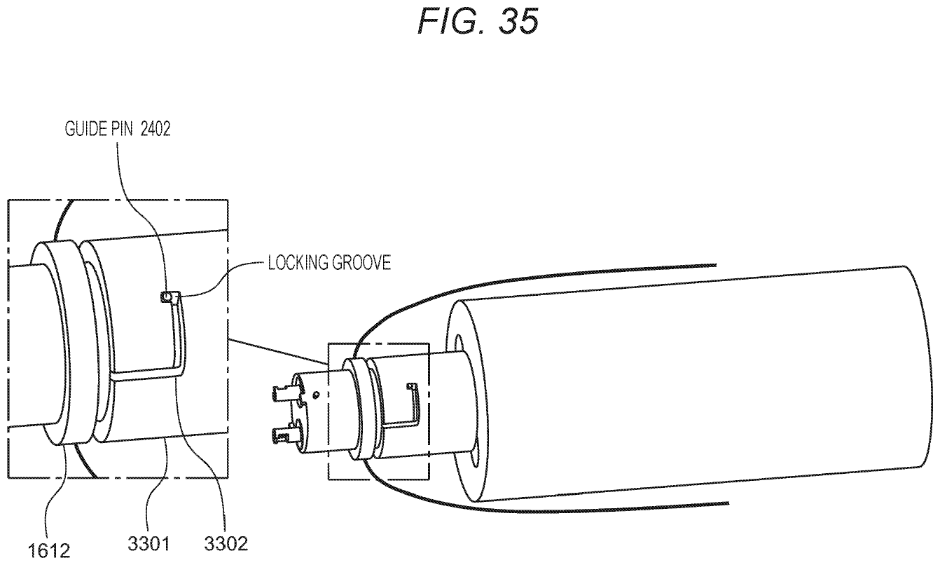

Then, the adapter 1002 can be attached to the drive unit 1003 by finally pushing the guide pin 2402 into the locking groove at the innermost part of the L-shaped groove 3302 using the reaction force of the spring attached to the side surface of the rib 1612 to lock the guide pin 2402 when reaching the toe of the L-shaped foot of the L-shaped groove 3302 (see FIG. 35). Since the guide pin 2402 of the adapter 1002 is locked, the detachment of the adapter 1002 from the drive unit 1003 is restricted.

### F-2. Attachment of Surgical Instrument Unit to Adapter

A procedure for attaching the surgical instrument unit 1001 to the adapter 1002 will be described with reference to FIGS. 36 to 39.

The surgical instrument unit 1001 is attached to the adapter 1002 is performed after the attachment of the adapter 1002 to the surgical instrument unit 1003 is completed according to the procedure described in the section F-1 described above. Therefore, at this point in time, each of the linear shafts B of the adapter 1002 is coupled with each of the corresponding linear shafts A on the drive unit 1003 side.

First, the actuator is driven in the drive unit 1003 to stop the linear shaft B coupled with the linear shaft A at an attachment and detachment position (see FIG. 36). The attachment and detachment position of the linear shaft B is a reference position in the longitudinal-axis direction of the linear shaft B for appropriately coupling corresponding end parts of the linear shaft B and linear shaft C when the adapter 1002 is attached to the drive unit 1003 and the surgical instrument unit 1001 is further attached to the adapter 1002. When the linear shaft A is set to the attachment and detachment position, the linear shaft B coupled with the linear shaft A is naturally set to the attachment and detachment position.

As described above, the linear L-shaped groove 2703, which has the L shape and advances in the circumferential direction by being bent at the right angle after advancing in the longitudinal-axis direction from the edge of the rear end, is formed in the tubular receiving part 2701 of the surgical instrument unit 1001. The L-shaped groove 2703 includes the locking groove bent in the longitudinal-axis direction at the toe of the L-shaped foot (see FIGS. 27 and 29). Furthermore, the guide pin 2401 protrudes from the side surface on the front end side (or distal end side) of the adapter 1002 (see FIG. 24).

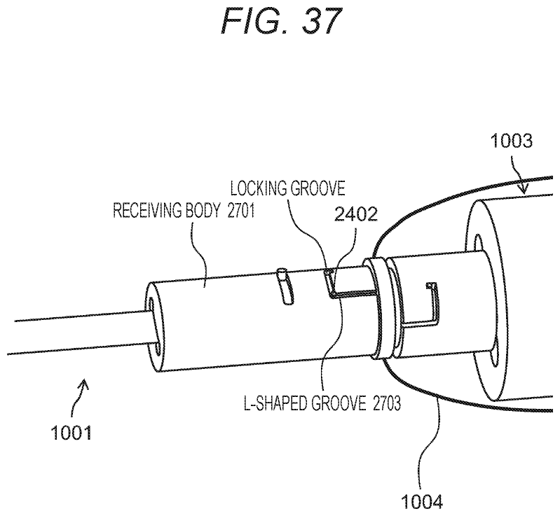

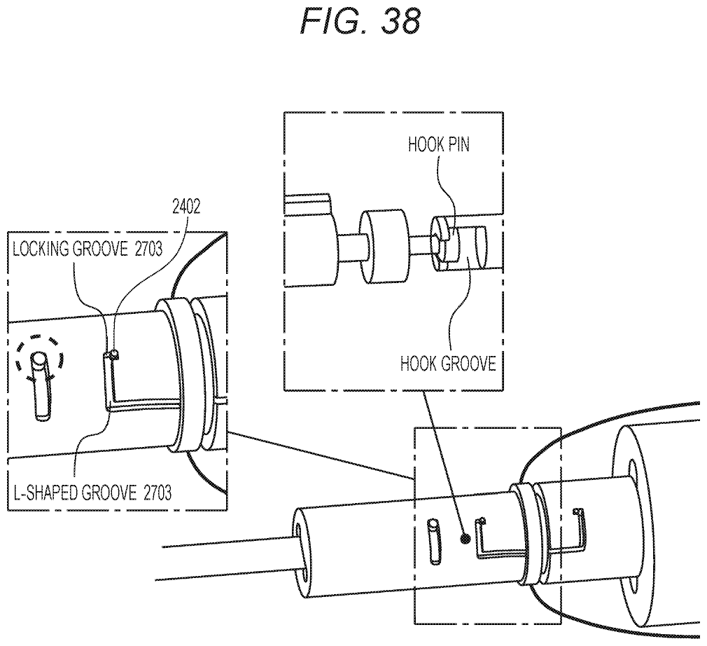

The locking device 2600 of the surgical instrument unit 1001 is locked, the guide pin 2401 is inserted into the L-shaped groove 2703 with the entrance of the L-shaped groove 2703 of the receiving part 2701 on the surgical instrument unit 1001 side being aligned with the guide pin 2401 on the adapter 1002 side, and the adapter 1002 is inserted into the receiving part 2701 in the longitudinal-axis direction such that the guide pin 2401 follows the L shape of the L-shaped groove 2703 (see FIG. 37).

When the guide pin 2401 reaches the bent portion of the L shape of the L-shaped groove 2703, the surgical instrument unit 1001 is then rotated about the longitudinal axis by about 45 degrees with respect to the adapter 1002. A rear end edge of the receiving part 2701 on the surgical instrument unit 1001 side just abuts on the rib 1612 of the adapter 1002 at a position where the guide pin 2401 reaches the bent portion of the L shape of the L-shaped groove 2703. As described with reference to FIG. 25, the hook pin is formed at the rear end of the linear shaft C, and the hook groove is formed at the front end of the linear shaft B. The hook groove has the opening that receives the hook pin and the U-shaped notch that locks the hook pin entering from the opening. When the guide pin 2401 reaches the bent portion of the L shape of the L-shaped groove 2703, the hook pin and the hook groove are positioned to just overlap each other, and the opening of the hook groove faces the hook pin. Then, the hook pin at the rear end of the linear shaft C is accommodated in the opening of the hook groove of the linear shaft B and is hooked by the U-shaped notch (see the left side of FIG. 38) in the course of rotation of the surgical instrument unit 1101 about the longitudinal axis by about 45 degrees with respect to the adapter 1002, whereby the linear shaft C is coupled with the linear shaft B. When the receiving part 2701 of the surgical instrument unit 1001 is attached to the adapter 1002 such that the guide pin 2401 is set along the L-shaped groove 2703, the hook pin at the rear end of the linear shaft C can be guided to follow a path of being hooked by the notch of the hook groove at the front end of the linear shaft B.

Then, the surgical instrument unit 1001 can be attached to the adapter 1002 by finally pushing the guide pin 2401 into the locking groove at the innermost part of the L-shaped groove 2703 using the reaction force of the spring attached to the side surface of the rib 1612 to lock the guide pin 2401 when reaching the toe of the L-shaped foot of the L-shaped groove 2703 (see FIG. 38). Since the guide pin 2601 of the adapter 1002 is locked, the detachment of the surgical instrument unit 1001 from the adapter 1002 is restricted.

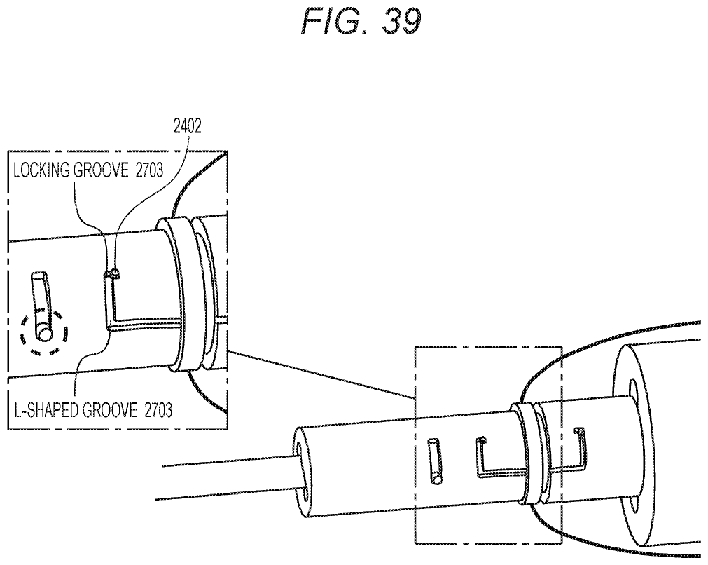

In this manner, when the attachment of the surgical instrument unit 1001 to the adapter 1002 is completed, the manipulating element 2604 protruding from the outer periphery of the locking device 2600 is manipulated along the groove 2702 in the circumferential direction of the receiving part 2701 to set the locking device 2600 of the surgical instrument unit 1001 in the lock-off state and release the restriction on the movement of the linear shaft C (see FIG. 39). As a result, the drive unit 1003 is set in an operable state capable of driving the end effector of the surgical instrument unit 1001 via the linear shaft B of the adapter 1002 as the translatory transmission part. Furthermore, since the guide pins 2401 and 2402 of the adapter 1002 are both locked by the reaction forces of the springs although the locking device 2600 is in the lock-off state, the detachment of the surgical instrument unit 1001 and the adapter 1002 is restricted.

F-3. Detachment of Surgical Instrument Unit from Adapter

A procedure for detaching the surgical instrument unit 1001 from the adapter 1002 will be described with reference to FIGS. 40 to 43.

The surgical instrument unit 1001, the adapter 1002, and the drive unit 1003 are in a state in which attachment is completed according to the procedures described in the sections F-1 and F-2 described above. Therefore, at this point in time, the corresponding linear shafts A to C are coupled with each other.

First, the actuator is driven in the drive unit 1003 to stop each of the sets of the linear shafts A to C coupled over the drive unit 1003, the adapter 1002, and the surgical instrument unit 1001 at the attachment and detachment position (see FIG. 40).

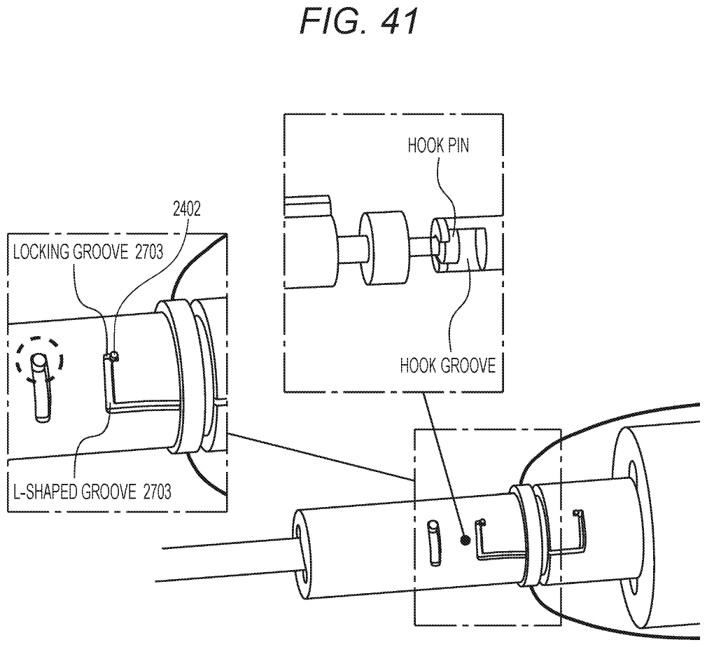

At this point in time, the locking device 2600 of the surgical instrument unit 1001 is in the lock-off state. First, the locking device 2600 is rotated in an opposite direction about the longitudinal axis using the manipulating element 2604 to switch to the lock-on state and fix the linear shaft C (see FIG. 41). Furthermore, since the guide pin 2401 is pushed into the locking groove at the innermost part of the L-shaped groove 2703 of the receiving part 2701 on the surgical instrument unit 1001 side and is in the locked state, the locked state of the guide pin 2401 is released. Since the guide pin 2401 is pushed into the locking groove by the reaction force of the spring attached to the side surface of the rib 1612, the guide pin 2401 can be taken out from the locking groove by once pushing the surgical instrument unit 1001 toward the adapter 1002, and further, the guide pin 2401 can be unlocked by reversely rotating the adapter 1002 about the longitudinal axis.

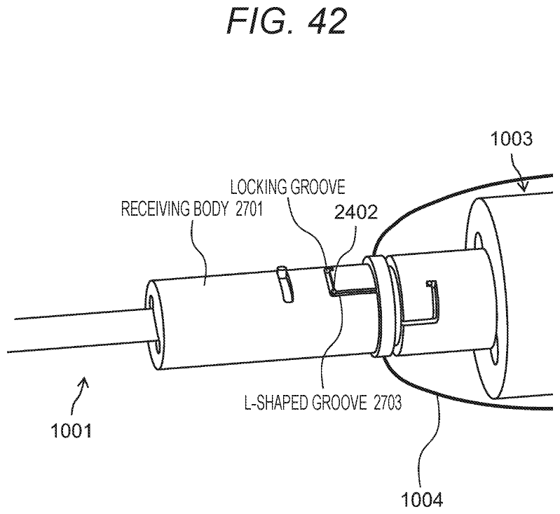

Next, the surgical instrument unit 1001 is rotated about the longitudinal axis by about 45 degrees with respect to the adapter 1002 in a direction opposite to that at the time of attachment. In the course of the reverse rotation by about 45 degrees, the hook pin at the rear end of the linear shaft C is released from the hook groove at the front end of the linear shaft B, and the linear shaft B and the linear shaft C are separated (see FIG. 42).

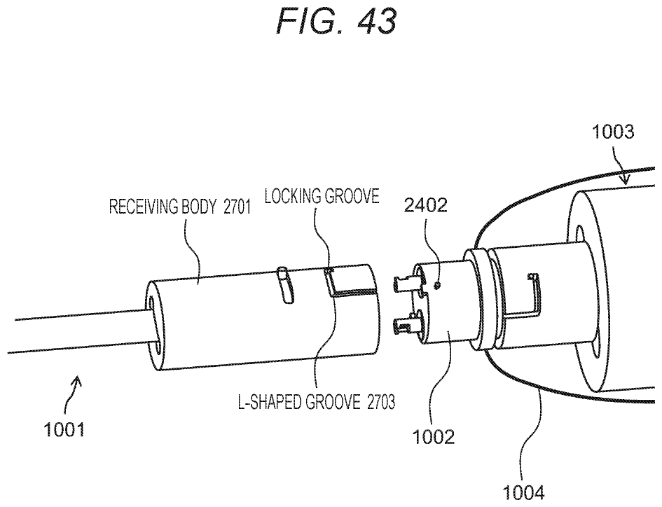

When the surgical instrument unit 1001 is reversely rotated about the longitudinal axis with respect to the adapter 1002 and the guide pin 2401 reaches the bent portion of the L shape of the L-shaped groove 2703, the surgical instrument unit 1001 is then pulled in the longitudinal direction from the adapter 1002 such that the guide pin 2401 follows the L-shape of the L-shaped groove 2703. Then, when the guide pin 2401 on the adapter 1002 side comes out of the entrance of the L-shaped groove 2703 of the receiving part 2701 on the surgical instrument unit 1001 side, the detachment of the surgical instrument unit 1001 from the adapter 1002 is completed (see FIG. 43).

F-4. Detachment of Adapter from Drive Unit

A procedure for detaching the adapter 1002 from the drive unit 1003 will be described with reference to FIGS. 44 to 47.

The adapter 1002 is detached from the drive unit 1003 after the surgical instrument unit 1001 is detached from the adapter 1002 according to the procedure described in the section F-3 described above. Therefore, each of the linear shafts B of the adapter 1002 is coupled with each of the corresponding linear shafts A on the drive unit 1003 side.

First, the actuator is driven in the drive unit 1003 to stop the linear shaft B coupled with the linear shaft A at an attachment and detachment position (see FIG. 44).

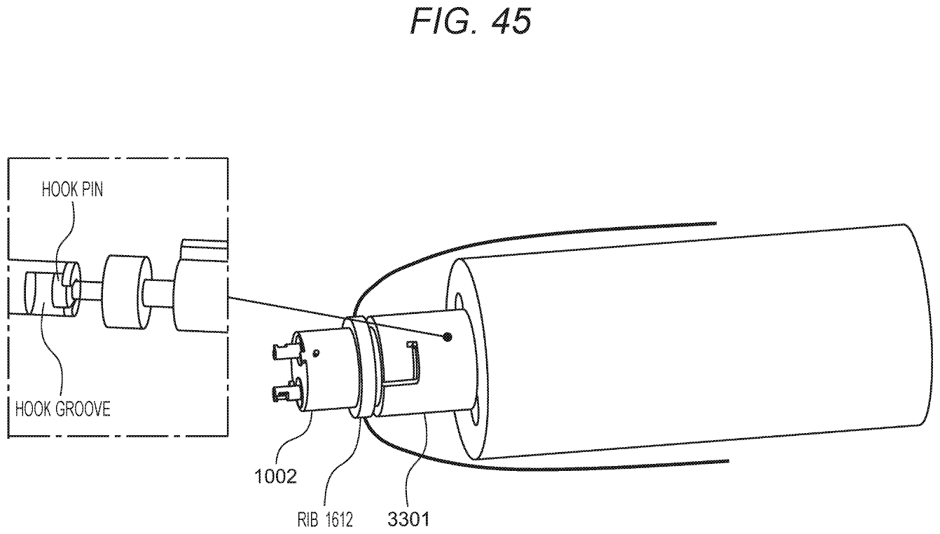

At this point in time, since the guide pin 2402 is pushed into the locking groove at the innermost part of the L-shaped groove 3302 of the receiving part 3301 on the drive unit 1003 side and is in the locked state, the locked state of the guide pin 2402 is released. Since the guide pin 2401 is pushed into the locking groove by the reaction force of the spring attached to the side surface of the rib 1612, the guide pin 2402 can be taken out from the locking groove by once pushing the adapter 1002 toward the drive unit 1003 (see FIG. 45).

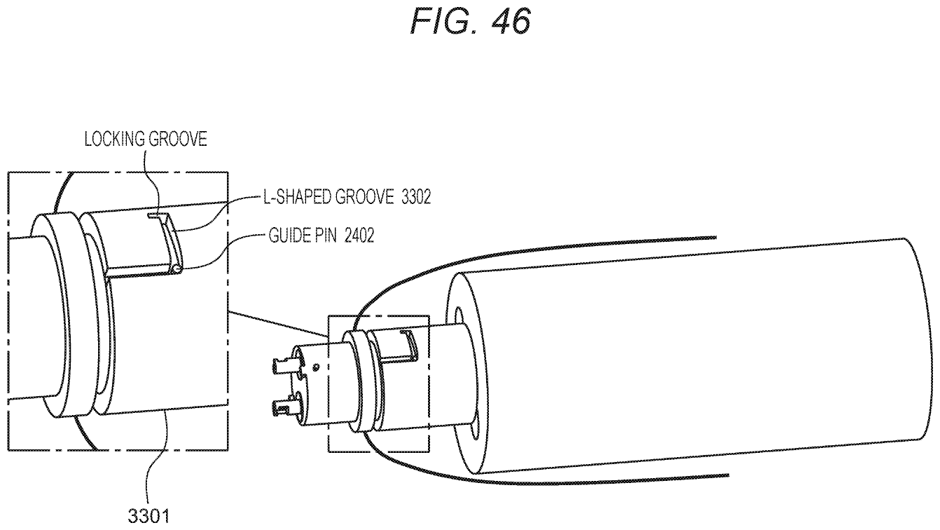

Next, the adapter 1002 is rotated about the longitudinal axis by about 45 degrees with respect to the drive unit 1003 in a direction opposite to that at the time of attachment (see FIG. 46). In the course of the reverse rotation by about 45 degrees, the hook pin at the front end of the linear shaft A is released from the hook groove at the rear end of the linear shaft B, and the linear shaft A and the linear shaft B are separated.

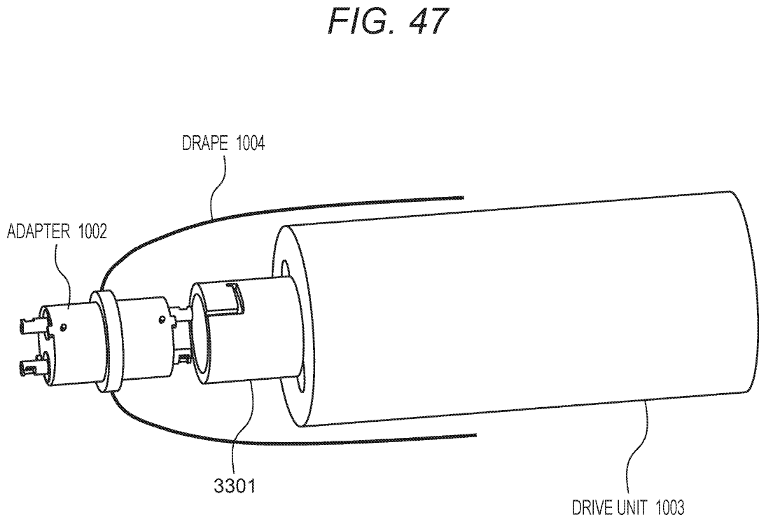

When the adapter 1002 is reversely rotated about the longitudinal axis with respect to the drive unit 1003 and the guide pin 2402 reaches the bent portion of the L shape of the L-shaped groove 3302, the surgical instrument unit 1001 is then pulled in the longitudinal direction from the adapter 1002 such that the guide pin 2402 follows the L-shape of the L-shaped groove 3302. Then, when the guide pin 2402 on the adapter 1002 side comes out of the entrance of the L-shaped groove 3302 of the receiving part 3301 on the drive unit 1003 side, the detachment of the adapter 1002 from the drive unit 1003 is completed (see FIG. 47).

### G. Modified Examples

In this section, modified examples of the surgical instrument device 1000 will be described.

### G-1. Regarding Number of Translatory Transmission Mechanisms

Although the description has been given in FIGS. 12 to 47 assuming that the number of wires (or translatory transmission mechanisms) is limited to two in order to prevent the complication of the drawings, but a similar translatory transmission structure can be added according to the number of wires to be actually used. For example, in the section D described above (see FIGS. 4 to 9), the configuration example in which the surgical instrument is driven by inserting the four wires (C1, C2, C3a and C3b) has been described. However, even if a total of four translatory transmission parts corresponding to the respective wires are provided, an adapter having a drip-proof structure can be manufactured to separate a surgical instrument unit in a clean region and a drive unit in an unclean region.

### G-2. Regarding Locking Device

It is assumed that the linear shaft A of the drive unit 1003 operates in both directions in the longitudinal-axis direction in the embodiment illustrated in FIGS. 12 to 47. However, in a case where it is assumed that the linear shaft A operates only in a direction of being pulled from the front end side, a locking device that fixes the linear shaft A when not in use may be provided similarly to the surgical instrument unit 1001.

### G-3. Regarding Adapter

The air chamber between the shield rubbers 1601 and 1602 is sealed in the state in which the preload is applied, but an air pipe may be connected from the outside to perform pressurization such that a constant air pressure is always maintained.

Although the air chamber is formed by the structure in which the shield rubber 1601 and the shield rubber 1602 each adopting the top hat type are symmetrically arranged such that their flanges face each other, one shield rubber of the top hat type may be used for one linear shaft B.

It is desirable fir the shield rubber having a two-fold structure to be smoothly deformed by rolling while changing the fold length with low friction and without oil supply, but a material is not particularly limited as long as this requirement is satisfied. For example, the shield rubber may be manufactured using a hybrid material of fiber mesh and rubber.

In a case where a plurality of the linear shafts B is used, the respective shield rubbers may be integrally molded.

### G-4. Regarding Actuator

The actuator that drives the linear shaft A in the drive unit 1003 can be exemplified as follows. In a case where a plurality of actuators is mounted, two or more types of actuators may be used in combination.
Electromagnetic rotary motor
Electromagnetic linear motor
Pneumatic cylinder
Water pressure cylinder
Hydraulic cylinder
Ultrasonic rotary motor
Ultrasonic linear motor

Furthermore, an actuator may be equipped with a speed reducer, a position detector, and an emergency brake mechanism regardless of which type of actuator is adopted. Here, examples of the speed reducer can include a gear-type speed reducer, a wave gear speed reducer, a planetary gear speed reducer, a paradox planetary gear speed reducer, a cable speed reducer, a traction speed reducer, a ball screw, a sliding screw, a worm gear, and the like. Furthermore, examples of the position detector can include a magnetic encoder, an optical encoder, and a potentiometer.

### G-5. Identification of Surgical Instrument Unit

The operation of replacing and using a plurality of types of surgical instrument units via an adapter is performed for one medical arm device or surgical manipulation device. Therefore, each of the surgical instrument units may be equipped with an identification device for specifying a surgical instrument type (forceps, a pneumoperitoneum tube, an energy treatment instrument, tweezers, a retractor, and the like).

The identification device needs to be readable from a drive unit (or the medical arm device or the surgical manipulation device on which the drive unit is mounted) via the adapter. The identification device may be, for example, an integrated circuit (IC) chip, but may express information such as a surgical instrument type by a two-dimensional barcode or a shape of a coupling portion (such as the tubular receiving part 2701). In a case where an IC chip is used, various types of information such as a shape, a weight, and the number of times of use of a surgical instrument may be recorded on the IC chip in addition to the surgical instrument type.

### G-6. Regarding Operation

The adapter may be disposable after one surgical operation, and the surgical instrument unit may be sterilized after use and reused up to a predetermined number of times. Alternatively, the adapter and the surgical instrument unit may be formed in an integrated structure and be sterilized after use and reused up to a predetermined number of times. Alternatively, the adapter and the surgical instrument unit may be formed in an integrated structure and be disposable after one surgical operation.

### H. Effects

Effects brought by the surgical instrument unit, the medical arm device, and the surgical manipulation device to which the present disclosure is applied will be summarized.
According to the present disclosure, the mechanism of the surgical instrument unit is simplified, and thus, the cost reduction can be achieved, and the sterilization processing is facilitated.
According to the present disclosure, since the surgical instrument unit is attached to the drive unit via the adapter having the drip-proof structure, the clean region and the unclean region can be completely separated, and reliability is improved.
According to the present disclosure, the adapter has the drip-proof structure of the translatory transmission part (linear shaft B) inserted through the through-hole achieved by using the shield rubber (rolling diaphragm) having the two-fold structure, and maintains the drip-proof property as the shield rubber is smoothly deformed by rolling with low friction and without oil supply while changing the fold length when the translatory transmission part takes the translatory movement. Therefore, the tight fitting tolerance for inserting the translatory transmission part is not required, and thus, the cost reduction can be achieved.
According to the drip-proof structure of the adapter of the present disclosure, even if blood adheres to a sliding part of the translatory transmission part, the translatory transmission part can be smoothly operated while maintaining the drip-proof property by the shield rubber, and thus, the reliability is improved.
With the drip-proof structure of the adapter according to the present disclosure, the shield rubber having the two-fold structure is smoothly deformed by rolling with low friction and without oil supply while changing the fold length, and thus, internal disturbance in the translatory transmission part decreases, and transparency regarding the operation of the surgical instrument is improved.

### INDUSTRIAL APPLICABILITY

The present disclosure has been described in detail above with reference to specific embodiments. However, it is self-evident that those skilled in the art can make modified examples and substitutions of the embodiments within a scope not departing from a gist of the present disclosure.

In the present specification, the embodiment in which the present disclosure is applied to the surgical robot to separate the clean region and the unclean region in the mechanism that attaches and detaches the surgical instrument unit to be sterilized from and to the drive unit has been mainly described, but the gist of the present disclosure is not limited thereto. The present disclosure can also be applied to fields other than medical care to achieve separation between a clean region and an unclean region in a mechanism that attaches and detaches two units, and complete separation of a region for each unit when two detachable units are attached.

In short, the present disclosure has been described in the form of exemplification, and the contents described in the present specification should not be interpreted in a limited manner. In order to determine the gist of the present disclosure, the scope of claims should be taken into consideration.

Note that the present disclosure can also have the following configurations.
(1) A medical manipulator system including:
   a surgical instrument unit that includes a surgical instrument at a front end;
   a drive unit that drives the surgical instrument; and
   an adapter that attaches the surgical instrument unit to the drive unit,
   in which the adapter includes: a translatory transmission part that transmits a driving force generated by the drive unit to the surgical instrument unit; and a drip-proof part that separates a side of the surgical instrument unit and a side of the drive unit in the translatory transmission part.
(2) The medical manipulator system according to (1) described above, in which
   the drip-proof part performs the separation by a structure in which an air chamber is provided between the side of the surgical instrument unit and the side of the drive unit in the translatory transmission part.
(3) The medical manipulator system according to (2) described above, in which
   the drip-proof part has a structure in which two elastic bodies each having a two-fold structure are arranged to face each other, and the air chamber is formed between the elastic bodies by connecting both ends with the translatory transmission part.
(4) The medical manipulator system according to (2) or (3) described above, in which
   the air chamber is maintained at a constant air pressure.
(5) The medical manipulator system according to according to any one of (1) to (3) described above, in which
   the drive unit includes a first linear shaft and an actuator that causes translatory movement of the first linear shaft,
   the adapter includes a second linear shaft as the translatory transmission part,
   the surgical instrument unit includes a third linear shaft, and
   the second linear shaft is coupled with the first linear shaft when the adapter is attached to the drive unit, the third linear shaft is coupled with the second linear shaft when the surgical instrument unit is attached to the adapter, and a driving force of the actuator is transmitted by the first, second, and third linear shafts to drive the surgical instrument.
(6) The medical manipulator system according to (5) described above, in which
   the surgical instrument unit further includes a locking device that restricts movement of the third linear shaft.
(7) The medical manipulator system according to (6) described above, in which
   the locking device has a locked state in which the operation of the third linear shaft is restricted and an unlocked state in which the restriction is released.
(8) The medical manipulator system according to (7) described above, in which
   the locking device is in the locked state when the attachment of the surgical instrument unit to the adapter is not completed.
(9) The medical manipulator system according to any one of (5) to (8) described above, in which
   the surgical instrument unit includes a receiving part of the adapter, the receiving part including a guiding part that guides the coupling between the third linear shaft and the second linear shaft.
(10) The medical manipulator system according to (9) described above, in which
   the guiding part guides operations of the receiving part and the adapter to follow a path along which a hook pin at an end part of the second linear shaft is hooked on a hook groove at an end part of the third linear shaft.
(11) The medical manipulator system according to (10) described above, in which
   the guiding part performs the guidance by causing a guide pin protruding from an outer periphery of the adapter to follow a specific shape of a groove formed in the receiving part.
(12) The medical manipulator system according to (11) described above, further including
   a locking groove which is provided at a terminal end of the groove and in which the guide pin is drawn.
(13) The medical manipulator system according to any one of (5) to (12) described above, in which
   the drive unit includes a receiving part of the adapter, the receiving part including a guiding part that guides the coupling between the first linear shaft and the second linear shaft.
(14) The medical manipulator system according to (13) described above, in which
   the guiding part guides operations of the receiving part and the adapter to follow a path along which a hook pin at an end part of the second linear shaft is hooked on a hook groove at an end part of the first linear shaft.
(15) The medical manipulator system according to (14) described above, in which
   the guiding part performs the guidance by causing a guide pin protruding from an outer periphery of the adapter to follow a specific shape of a groove formed in the receiving part.
(16) The medical manipulator system according to (15) described above, further including
   a locking groove which is provided at a terminal end of the groove and in which the guide pin is drawn
(17) An adapter device having one end to which a drive unit is attached and another end to which a surgical instrument unit is attached, the adapter device including:
   a translatory transmission part that transmits a driving force generated by the drive unit to the surgical instrument unit; and
   a drip-proof part that separates a side of the surgical instrument unit and a side of the drive unit in the translatory transmission part.
(18) The adapter device according to (17) described above, in which
   the drip-proof part performs the separation by a structure in which an air chamber is provided between the side of the surgical instrument unit and the side of the drive unit in the translatory transmission part.
(19) The adapter device according to (18) described above, in which
   the drip-proof part has a structure in which two elastic bodies each having a two-fold structure are arranged to face each other, and the air chamber is formed between the elastic bodies by connecting both ends with the translatory transmission part.
(20) The adapter device according to any one of claims 17 to 19, further including
   a second linear shaft, as the translatory transmission part, having both ends coupled with a first linear shaft of the drive unit and a third linear shaft of the surgical instrument unit, respectively.

### REFERENCE SIGNS LIST

100 Surgical support system
110 Medical arm device
111 Active joint part
111A Actuator
111B Torque sensor
111C Encoder
112 Passive joint part
112A Encoder
113 Sensor part
120 Control device
130 Input device
210 Arm
220 Front end part
221 Surgical instrument unit
222 Drive unit
300 Surgical manipulation device
310 Handle part
311 Joystick
312 Button
320 Front end part
321 Surgical instrument unit
322 Drive unit
400 Surgical instrument unit
401 Opening and closing mechanism
402 Shaft
403 Drive unit
1000 Surgical instrument device
1001 Surgical instrument unit
1002 Adapter
1003 Drive unit
1004 Drape
1401, 1402 Actuator
1601, 1602 Shield rubber
1611 Through-hole
1612 Rib
2401 Guide pin (for attachment to surgical instrument unit 1001)
2402 Guide pin (for attachment to drive unit 1003)
2403, 2404 Protrusion
2600 Locking device
2601 Bottom surface
2602 Locking claw
2603 Release part
2604 Manipulating element
2605, 2606 Recess
2701 Receiving part
2702 Groove
2703 L-shaped groove
3101 Constricted part
3301 Receiving part
3302 L-shaped groove

1. A medical manipulator system comprising:
a surgical instrument unit that includes a surgical instrument at a front end;
a drive unit that drives the surgical instrument; and
an adapter that attaches the surgical instrument unit to the drive unit,
wherein the adapter includes: a translatory transmission part that transmits a driving force generated by the drive unit to the surgical instrument unit; and a drip-proof part that separates a side of the surgical instrument unit and a side of the drive unit in the translatory transmission part.

2. The medical manipulator system according to claim 1, wherein
the drip-proof part performs the separation by a structure in which an air chamber is provided between the side of the surgical instrument unit and the side of the drive unit in the translatory transmission part.

3. The medical manipulator system according to claim 2, wherein
the drip-proof part has a structure in which two elastic bodies each having a two-fold structure are arranged to face each other, and the air chamber is formed between the elastic bodies by connecting both ends by the translatory transmission part.

4. The medical manipulator system according to claim 2, wherein
the air chamber is maintained at a constant air pressure.

5. The medical manipulator system according to claim 1, wherein
the drive unit includes a first linear shaft and an actuator that causes translatory movement of the first linear shaft,
the adapter includes a second linear shaft as the translatory transmission part,
the surgical instrument unit includes a third linear shaft, and
the second linear shaft is coupled with the first linear shaft when the adapter is attached to the drive unit, the third linear shaft is coupled with the second linear shaft when the surgical instrument unit is attached to the adapter, and a driving force of the actuator is transmitted by the first, second, and third linear shafts to drive the surgical instrument.

6. The medical manipulator system according to claim 5, wherein
the surgical instrument unit further includes a locking device that restricts movement of the third linear shaft.

7. The medical manipulator system according to claim 6, wherein
the locking device has a locked state in which the operation of the third linear shaft is restricted and an unlocked state in which the restriction is released.

8. The medical manipulator system according to claim 7, wherein
the locking device is in the locked state when the attachment of the surgical instrument unit to the adapter is not completed.

9. The medical manipulator system according to claim 5, wherein
the surgical instrument unit includes a receiving part of the adapter, the receiving part including a guiding part that guides the coupling between the third linear shaft and the second linear shaft.

10. The medical manipulator system according to claim 9, wherein
the guiding part guides operations of the receiving part and the adapter to follow a path along which a hook pin at an end part of the second linear shaft is hooked on a hook groove at an end part of the third linear shaft.

11. The medical manipulator system according to claim 10, wherein
the guiding part performs the guidance by causing a guide pin protruding from an outer periphery of the adapter to follow a specific shape of a groove formed in the receiving part.

12. The medical manipulator system according to claim 11, further comprising
a locking groove which is provided at a terminal end of the groove and in which the guide pin is drawn.

13. The medical manipulator system according to claim 5, wherein
the drive unit includes a receiving part of the adapter, the receiving part including a guiding part that guides the coupling between the first linear shaft and the second linear shaft.

14. The medical manipulator system according to claim 13, wherein
the guiding part guides operations of the receiving part and the adapter to follow a path along which a hook pin at an end part of the second linear shaft is hooked on a hook groove at an end part of the first linear shaft.

15. The medical manipulator system according to claim 14, wherein
the guiding part performs the guidance by causing a guide pin protruding from an outer periphery of the adapter to follow a specific shape of a groove formed in the receiving part.

16. The medical manipulator system according to claim 15, further comprising
a locking groove which is provided at a terminal end of the groove and in which the guide pin is drawn.

17. An adapter device having one end to which a drive unit is attached and another end to which a surgical instrument unit is attached, the adapter device comprising:
a translatory transmission part that transmits a driving force generated by the drive unit to the surgical instrument unit; and
a drip-proof part that separates a side of the surgical instrument unit and a side of the drive unit in the translatory transmission part.

18. The adapter device according to claim 17, wherein
the drip-proof part performs the separation by a structure in which an air chamber is provided between the side of the surgical instrument unit and the side of the drive unit in the translatory transmission part.

19. The adapter device according to claim 18, wherein
the drip-proof part has a structure in which two elastic bodies each having a two-fold structure are arranged to face each other, and the air chamber is formed between the elastic bodies by connecting both ends with the translatory transmission part.

20. The adapter device according to claim 17, further comprising
a second linear shaft, as the translatory transmission part, having both ends coupled with a first linear shaft of the drive unit and a third linear shaft of the surgical instrument unit, respectively.
